(19) United States
(12) Patent Application Publication
(10) Pub. No.: US 2006/0142312 A1
(43) Pub. Date: Jun. 29, 2006

(54) C6-aryl and heteroaryl substituted pyrido[2,3-D] pyrimidin-7-ones

(75) Inventors: Cathlin Marie Flamme, Lawrenceville, NJ (US); Douglas S. Johnson, Dexter, MI (US); Dennis Joseph McNamara, Ann Arbor, MI (US); Debra Ann Sherry, Chelsea, MI (US); Peter Laurence Toogood, Ann Arbor, MI (US); Scott Norman VanderWel, Ann Arbor, MI (US)

Correspondence Address: AGOURON PHARMACEUTICALS, INC., 10777 SCIENCE CENTER DRIVE, SAN DIEGO, CA, 92121, US

(73) Assignee: PFIZER INC

(21) Appl. No.: 11/021,037

(22) Filed: Dec. 23, 2004

(51) Int. Cl. A61K 31/675; A61K 31/519

(52) U.S. Cl. 514/264.1; 514/264.11; 544/279; 514/81; 544/244

(57) ABSTRACT

The present invention provides substituted 2-aminopyridines of formula I, wherein R1, A1, W, X, and Y are as defined in the specification, useful in treating cell proliferative disorders. The novel compounds of the present invention are potent inhibitors of cyclin-dependent kinases 4 (Cdk4).

### FIELD OF THE INVENTION

This invention relates to substituted C6-substituted pyrido[2,3-d]pyrimidines that are potent inhibitors of cyclin-dependent kinase 4. The compounds of the invention are useful for the treatment of inflammation, and cell proliferative diseases such as cancer and restenosis.

### SUMMARY OF THE RELATED ART

Cyclin-dependent kinases and related serine/threonine protein kinases are important cellular enzymes that perform essential functions in regulating cell division and proliferation. The cyclin-dependent kinase catalytic units are activated by regulatory subunits known as cyclins. At least 16 mammalian cyclins have been identified (Johnson D. G. and Walker C. L., Annu. Rev. Pharmacol. Toxicol. 1999; 39:295-312) as well as 11 cyclin-dependent kinases Manning, G. et al. Science 2002, 298, 1912-1934). Cyclin B/Cdk1, Cyclin A/Cdk2, Cyclin E/Cdk2, Cyclin D/Cdk4, Cyclin D/Cdk6, and probably other heterodimers including Cdk3 and Cdk7 are important regulators of cell cycle progression. Additional functions of Cyclin/Cdk heterodimers include regulation of transcription, DNA repair, differentiation and apoptosis (Morgan D. O., Annu. Rev. Cell. Dev. Biol. 1997; 13261-13291).

Increased activity or temporally abnormal activation of cyclin-dependent kinases has been shown to result in the development of human tumors (Sherr C. J., Science 1996; 274:1672-1677). Indeed, human tumor development is commonly associated with alterations in either the Cdk proteins themselves or their regulators (Cordon-Cardo C., Am. J. Pathol. 1995; 147:545-560; Karp J. E. and Broder S., Nat. Med. 1995; 1:309-320; Hall M. et al., Adv. Cancer Res. 1996; 68:67-108). Naturally occurring protein inhibitors of Cdks such as p16 and p27 cause growth inhibition in vitro in lung cancer cell lines (Kamb A., Curr. Top. Microbiol. Immunol. 1998; 227:139-148).

Small molecule Cdk inhibitors may also be used in the treatment of cardiovascular disorders such as restenosis and atherosclerosis and other vascular disorders that are due to aberrant cell proliferation. Vascular smooth muscle proliferation and intimal hyperplasia following balloon angioplasty are inhibited by over-expression of the cyclin-dependent kinase inhibitor protein. Moreover, the purine Cdk2 inhibitor CVT-313 (Ki=95 nM) resulted in greater than 80% inhibition of neointima formation in rats.

Cdk inhibitors can be used to treat diseases caused by a variety of infectious agents, including fungi, protozoan parasites such as Plasmodium falciparum, and DNA and RNA viruses. For example, cyclin-dependent kinases are required for viral replication following infection by herpes simplex virus (HSV) (Schang L. M. et al., J. Virol. 1998; 72:5626) and Cdk homologs are known to play essential roles in yeast.

Selective Cdk inhibitors can be used to ameliorate the effects of various autoimmune disorders. The chronic inflammatory disease rheumatoid arthritis is characterized by synovial tissue hyperplasia; inhibition of synovial tissue proliferation should minimize inflammation and prevent joint destruction. In a rat model of arthritis, joint swelling was substantially inhibited by treatment with an adenovirus expressing a Cdk inhibitor protein p.16. Cdk inhibitors are effective against other disorders of cell proliferation including psoriasis (characterized by keratinocyte hyperproliferation), glomerulonephritis, and lupus.

Certain Cdk inhibitors are useful as chemoprotective agents through their ability to inhibit cell cycle progression of normal untransformed cells (Chen, et al. J. Natl. Cancer Institute, 2000; 92:1999-2008). Pre-treatment of a cancer patient with a Cdk inhibitor prior to the use of cytotoxic agents can reduce the side effects commonly associated with chemotherapy. Normal proliferating tissues are protected from the cytotoxic effects by the action of the selective Cdk inhibitor.

Review articles on small molecule inhibitors of cyclin dependent kinases have noted the difficulty of identifying compounds that inhibit specific Cdk proteins without inhibiting other enzymes. Thus, despite their potential to treat a variety of diseases, no Cdk inhibitors are currently approved for commercial use (Fischer, P. M., Curr. Opin. Drug Discovery 2001, 4, 623-634; Fry, D. W. & Garrett, M. D. Curr. Opin. Oncologic, Endocrine & Metabolic Invest. 2000, 2, 40-59; Webster, K. R. & Kimball, D. Emerging Drugs 2000, 5, 45-59; Sielecki, T. M. et al. J. Med. Chem. 2000, 43, 1-18.).

WO 98/33798 discloses a class of pyrido[2,3-d]pyrimidines that display selectivity for Cdks versus other protein kinases. These compounds are distinct from the 6-aryl-pyrido[2,3-d]pyrimidines (WO 96/15128; WO 96/34867), which display the opposite selectivity, inhibiting tyrosine kinases in preference to cyclin-dependent kinases. Moreover, they represent a new structural class when compared to either the pyrimidines and 3,4-dihydropyrimidines of international patent application WO 99/61444 or the naphthyridones described in WO 99/09030.

WO 01/70741 disclosed one class of compounds, 5-alkyl-pyrido[2,3-d]pyrimidines, that exhibit selectivity for Cdk4 inhibition. A further class of Cdk4 selective compound was disclosed in U.S. patent application Ser. No. 10/345,778). However, there exists a need for other small molecular weight, highly selective inhibitors of Cdk4 that are orally bioavailable and useful for treating a wide variety of cell proliferative diseases and disorders, cancer, infections, autoimmune diseases, gout, kidney disease, and neurodegenerative diseases and disorders.

### SUMMARY OF THE INVENTION

This invention provides compounds of the formula I: 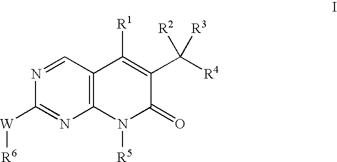
wherein:

W is selected from the group consisting of NH, N(CO)R⁷, NR⁷, S, S(O), S(O)₂, and halogen;

R¹ is hydrogen, C₁-C₆ alkyl, halogen, OR⁷, or NR⁷R⁸;

R² and R³ are independently selected from the group consisting of hydrogen, halogen, or C₁-C₆ alkyl, or R², R³ and the carbon to which they are attached may form a carbonyl group (C═O), or a C₃-C₅ carbocyclic ring;

R⁴ is a C₆-C₁₀ monocyclic or bicyclic aryl group or a 5 or 6 membered heteroaryl group wherein said aryl or heteroaryl group is optionally substituted with up to 5 substituents independently selected from halogen, C₁-C₆ alkyl, C₁-C₆ alkoxy, C₁-C₈ alkoxyalkyl, C₁-C₈ haloalkyl, C₁-C₈ hydroxyalkyl, C₂-C₈ alkenyl, C₂-C₈ alkynyl nitrile, and nitro; wherein said heteroaryl ring is selected from pyrrolyl, thienyl, furanyl, thiazolyl, triazolyl, imidazolyl, (is)oxazolyl, oxadiazolyl, tetrazolyl, pyridyl, thiadiazolyl, oxadiazolyl, oxathiadiazolyl, thiatriazolyl, pyrimidinyl, quinolinyl, isoquinolinyl, napthyridinyl, phthalimidyl, benzimidazolyl, and benzoxazolyl;

R⁵ is hydrogen, aryl, C₁-C₈ alkyl, C₁-C₈ alkoxy, C₃-C₇ cycloalkyl, C₃-C₈ alkenyl, C₅-C₈ cycloalkenyl, C₆-C₁₀ aryl, C₅-C₁₀ heteroaryl, or C₃-C₇-heterocyclyl;

R⁶ is hydrogen, (CR⁷R⁸)ₙAr, (CR⁷R⁸)ₙheteroaryl, (CR⁷R⁸)ₙheterocyclyl, C₁-C₁₀ alkyl, C₃-C₁₀ cycloalkyl, C₂-C₁₀ alkenyl, or C₂-C₁₀ alkynyl, wherein each of the (CR⁷R⁸)ₙAr, (CR⁷R⁸)ₙheteroaryl, alkyl, cycloalkyl, alkenyl, and alkynyl groups are optionally substituted with up to 7 groups selected from halogen, C₁-C₈ alkyl, C₃-C₇ cycloalkyl, C₃-C₈ cycloalkyloxy, C₃-C₈ heterocyclyl, C₃-C₈ heterocycloalkyloxy, C₃-C₈ heterocyclylalkyl, C₁-C₈ alkoxy, C₁-C₈ alkoxyalkyl, C₁-C₈ haloalkyl, C₁-C₈ hydroxyalkyl, C₂-C₈ alkenyl, C₂-C₈ alkynyl, phenoxy, phenyl, (CR⁹R¹⁰)ₙAr, (CR⁹R¹⁰)ₙheteroaryl, nitrile, nitro, (CR⁷R⁸)ₘR⁹, OR⁷, SR⁷, NR⁷R⁸, N(O)R⁷R⁸, P(O)(OR⁷)(OR⁸), (CR⁷R⁸)ₘNR⁹R¹⁰, (CR⁷R⁸)ₘC(O)N⁹R¹⁰, (CR⁷R⁸)ₘOR⁹, (CR⁷R⁸)ₘC(O)R⁹, (CR⁷R⁸)ₘCO₂R⁹, CONR⁷R⁸, C(O)NR⁷SO₂R⁸, NR⁷SO₂R⁸, C(O)NR⁷OR⁸, (CR⁷R⁸)ₘS(O)ₙR⁹, SO₂NR⁷R⁸, NR⁷CO₂R⁸, NR⁷COR⁸, NR⁷CONR⁸R⁹, NR⁷SO₂R⁸, N(O)R⁷R⁸, NR⁷R⁸R⁹Y, (CR⁷R⁸)ₘP(O)(OR⁹)(OR¹⁰), -T(CH₂)ₘQR⁷, —C(O)T(CH₂)ₘQR⁷, -T(CH₂)ₘC(O)NR⁷R⁸, -T(CH₂)ₘCO₂R⁷, and NR⁷C(O)T(CH₂)ₘQR⁷; wherein R⁶ optionally may be absent when W is a halogen;

n is an integer of from 1 to 3;

m is an integer of from 0 to 5;

T is selected from the group consisting of O, S, NR⁹, N(O)R⁹, and CR⁹R¹⁰;

Q is selected from the group consisting of O, S, NR⁹, N(O)R⁹, CO₂, O(CH₂)ₘ-heteroaryl, O(CH₂)ₘS(O)ₙR⁹, (CH₂)-heteroaryl, and a saturated or unsaturated carbocyclic group containing from 3-7 ring members, up to four of which ring carbon atoms are optionally replaced by heteroatoms independently selected from oxygen, sulfur, and nitrogen, provided, however, that there is at least one carbon atom in the carbocyclic ring and that, if there are two or more ring oxygen atoms, the ring oxygen atoms are not adjacent to one another, wherein the heteroaryl or carbocyclic group is unsubstituted or substituted with one, two, or three groups independently selected from halogen, hydroxy, hydroxyalkyl, C₁-C₈ alkyl, C₁-C₈ alkoxy, alkoxycarbonyl, alkylcarbonyl, alkylcarbonylamino, aminoalkyl, trifluoromethyl, N-hydroxyacetamide, trifluoromethylalkyl, amino, and mono or dialkylamino;

R⁷, R⁸, R⁹ and R¹⁰ are, in each instance, independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl;

or R⁷ and R⁸, when attached to the same nitrogen atom, taken together with the nitrogen to which they are attached, may form a saturated or unsaturated heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁹SO₂R¹⁰, C(O)NR⁹R¹⁰, NR⁹C(O)R¹⁰, C(O)OR⁹, C(O)NR⁹SO₂R¹⁰, (CH₂)ₘS(O)ₙR⁹, (CH₂)ₘ-heteroaryl, O(CH₂)ₘ-heteroaryl, (CH₂)ₘC(O)NR⁹R¹⁰, O(CH₂)ₘC(O)OR⁹, and (CH₂)SO₂NR⁹R¹⁰;

and the pharmaceutically acceptable salts, esters, amides, and prodrugs thereof.

The pyrido[2,3d]pyrimidines substituted at the carbon in the 6 position of the pyrido[2,3d]pyrimidine ring of Formula I and their pharmaceutically acceptable salts are useful for treating uncontrolled cell proliferative diseases, including, but not limited to, proliferative diseases such as cancer, restenosis and rheumatoid arthritis. In addition, these compounds and salts thereof are useful for treating inflammation and inflammatory diseases. In addition, these compounds and salts thereof have utility as antiinfective agents. Moreover, these compounds and salts thereof have utility as chemoprotective agents. The compounds of Formula I and salts thereof are selective for the serine/threonine kinases, cyclin-kinase, dependent kinase 4 and cyclin-dependent kinase 6. These compounds and salts thereof are readily synthesized and can be administered to patients by a variety of methods.

Compounds of Formula I may contain chiral centers and therefore may exist in different enantiomeric and diastereomeric forms. This invention relates to all optical isomers and all stereoisomers of compounds of the Formula I, both as racemic mixtures and as individual enantiomers and diastereoisomers of such compounds, and mixtures thereof, and to all pharmaceutical compositions and methods of treatment defined above that contain or employ them, respectively.

As the compounds of Formula I of this invention may possess asymmetric centers, they are capable of occurring in various stereoisomeric forms or configurations. Hence, the compounds can exist in separated (+)- and (−)-optically active forms, as well as mixtures thereof. The present invention includes all such forms within its scope. Individual isomers can be obtained by known methods, such as optical resolution, optically selective reaction, or chromatographic separation in the preparation of the final product or its intermediate.

The compounds of the present invention can exist in unsolvated forms as well as solvated forms, including hydrated forms. In general, the solvated forms, including hydrated forms, are equivalent to unsolvated forms and are intended to be encompassed within the scope of the present invention.

The present invention also includes isotopically labelled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

The compounds of Formula I are capable of further forming pharmaceutically acceptable formulations comprising salts, including but not limited to acid addition and/or base salts and solvates of a compound of Formula I.

This invention also provides pharmaceutical formulations comprising a therapeutically effective amount of a compound of Formula I or a therapeutically acceptable salt thereof and a pharmaceutically acceptable carrier, diluent, or excipient therefor. All of these forms are within the present invention.

By “alkyl,” in the present invention is meant a straight or branched hydrocarbon radical having from 1 to 10 carbon atoms, preferably 1 to 8 carbon atoms and includes, for example, methyl, ethyl, n-propyl, isopropyl, n-butyl, sec-butyl, isobutyl, tert-butyl, n-pentyl, iso-pentyl, n-hexyl, and the like.

“Alkenyl” means straight and branched hydrocarbon radicals having from 2 to 8 carbon atoms and at least one double bond and includes, but is not limited to, ethenyl, 3-buten-1-yl, 2-ethenylbutyl, 3-hexen-1-yl, and the like. The term “alkenyl” includes, cycloalkenyl, and heteroalkenyl in which 1 to 3 heteroatoms selected from O, S, N or substituted nitrogen may replace carbon atoms.

“Alkynyl” means straight and branched hydrocarbon radicals having from 2 to 8 carbon atoms and at least one triple bond and includes, but is not limited to, ethynyl, 3-butyn-1-yl, propynyl, 2-butyn-1-yl, 3-pentyn-1-yl, and the like.

“Cycloalkyl” means a monocyclic or polycyclic hydrocarbyl group having from 3 to 8 carbon atoms, for instance, cyclopropyl, cycloheptyl, cyclooctyl, cyclodecyl, cyclobutyl, adamantyl, norpinanyl, decalinyl, norbornyl, cyclohexyl, and cyclopentyl. Also included are rings in which 1 to 3 heteroatoms replace carbons. Such groups are termed “heterocyclyl,” which means a cycloalkyl group also bearing at least one heteroatom selected from O, S, N or substituted nitrogen. Examples of such groups include, but are not limited to, oxiranyl, pyrrolidinyl, piperidyl, tetrahydropyran, and morpholine.

By “alkoxy,” is meant straight or branched chain alkyl groups having 1-10 carbon atoms and linked through oxygen. Examples of such groups include, but are not limited to, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy. In addition, alkoxy refers to polyethers such as —O—(CH₂)₂—O—CH₃, and the like.

“Acyl” means an alkyl or aryl (Ar) group having from 1-10 carbon atoms bonded through a carbonyl group, i.e., R—C(O)—. For example, acyl includes, but is not limited to, a C₁-C₆ alkanoyl, including substituted alkanoyl, wherein the alkyl portion can be substituted by NR⁸R⁹ or a carboxylic or heterocyclic group. Typical acyl groups include acetyl, benzoyl, and the like.

The alkyl, alkenyl, alkoxy, and alkynyl groups described above are optionally substituted, preferably by 1 to 3 groups selected from NR⁸R⁹, phenyl, substituted phenyl, keto, amino, alkyl, thio C₁-C₆ alkyl, C₁-C₆ alkoxy, hydroxy, carboxy, C₁-C₆ alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₚPh where p is 1, 2, or 3. Perhalo and polyhalo substitution is also included.

Examples of substituted alkyl groups include, but are not limited to, 2-aminoethyl, 2-hydroxyethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, and 2-(4-methylpiperazinyl)ethyl.

Examples of substituted alkynyl groups include, but are not limited to, 2-methoxyethynyl, 2-ethylsulfanylethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include, but are not limited to, dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The term “anion” means a negatively charged counterion such as chloride, bromide, and trifluoroacetate.

The term “aryl”, as used herein, unless otherwise indicated, includes a C₆-C₁₀ aromatic ring system with no heteroatoms having a single ring (e.g., phenyl), multiple rings (e.g., biphenyl), or multiple fused rings in which at least one is aromatic, (e.g., 1,2,3,4-tetrahydronaphthyl, naphthyl, anthryl, or phenanthryl), wherein each aromatic ring in said aryl ring system can be optionally substituted with from one to three substituents independently selected from halogen, lower alkyl, lower alkoxy, lower alkylthio, trifluoromethyl, lower acyloxy, carbocyclic, heteroaryl, and hydroxy. A preferred aryl is phenyl which can be either unsubstituted or substituted with one, two or three substituents selected from the group consisting of halo, (C₁-C₄)alkyl optionally substituted with from one to three halogen atoms and (C₁-C₄)alkoxy optionally substituted with from one to three halogen atoms. The term “aryloxy”, as used herein, unless otherwise indicated, means “aryl-O—”, wherein “aryl” is as defined above.

The term “heteroaryl”, as used herein, unless otherwise indicated, includes an aromatic heterocycle containing five to ten ring members, of which from 1 to 4 can be heteroatoms selected, independently, from N, S and O, and which rings can be unsubstituted, monosubstituted or disubstituted with substituents selected, independently, from the group consisting of halo, (C₁-C₄)alkyl, and (C₁-C₄)alkoxy, said alkyl and alkoxy groups being optionally substituted with from one to three halogen atoms. Such heteroaryl groups include, but are not limited to, thienyl, furanyl, thiazolyl, triazolyl, imidazolyl, isoxazolyl, oxadiazolyl, tetrazolyl, pyridyl, pyrrolyl, thiadiazolyl, oxadiazolyl, oxathiadiazolyl, thiatriazolyl, pyrimidinyl, isoquinolinyl, quinolinyl, napthyridinyl, phthalimidyl, benzimidazolyl, and benzoxazolyl. A preferred heteroaryl is pyridine.

The term “heteroaryloxy”, as used herein, unless otherwise indicated, means “heteroaryl-O”, wherein heteroaryl is as defined above.

The term “leaving group”, as used herein, refers to any group (X) that can depart from the carbon to which it is attached carrying with it the two electrons that comprise the bond between the leaving group and that carbon (the X—C bond). Typical leaving groups include but are not limited to: halides (e.g. F⁻, Cl⁻, Br⁻, I⁻), esters, (e.g. acetate), sulfonate esters (e.g. mesylate, tosylate), ethers (EtO⁻, PhO⁻), sulfides (PhS⁻, MeS⁻), sulfoxides, and sulfones.

The term “one or more substituents”, as used herein, refers to a number of substituents that equals from one to the maximum number of substituents possible based on the number of available bonding sites.

By the terms “halo” or “halogen” in the present invention is meant fluorine, bromine, chlorine, and iodine.

The term “cancer” includes, but is not limited to, the following cancers: cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's Disease, hairy cell leukemia, and other leukemias.

The term “treating”, as used herein, refers to reversing, alleviating, inhibiting the progress of, or preventing the disorder or condition to which such term applies, or preventing one or more symptoms of such condition or disorder. The term “treatment”, as used herein, refers to the act of treating, as “treating” is defined immediately above. The term “treating” as used herein may be applied to any suitable mammal. Such mammals include, but are not limited to, canines, felines, bovines, ovines, equines, humans and the like.

The term “pharmaceutically acceptable salts, esters, amides, and prodrugs” as used herein refers to those salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

The term “salts” refers to the relatively non-toxic, inorganic and organic acid or base addition salts of compounds of the present invention. These salts can be prepared in situ during the final isolation and purification of the compounds or by separately reacting the purified compound in its free base or free acid form with a suitable organic or inorganic acid or base and isolating the salt thus formed. In so far as the compounds of formula I of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the basic compounds of Formula I are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Such acid addition salts may be prepared from inorganic acids. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like.

Such acid addition salts may also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

Pharmaceutically acceptable base addition salts can be formed from acidic compounds of the Formula I. Such salts are formed with metals or amines, such as alkali and alkaline earth metals, or organic amines. The base addition salts of acidic compounds of Formula I are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

Pharmaceutically acceptable base addition salts may include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, N,N-dibenzylethylenediamine, chloroprocaine, choline, diethanolamine, ethylenediamine, N-methylglucamine, and procaine and the like; see, for example, Berge et al., supra. Also contemplated are the salts of amino acids such as arginate, gluconate, galacturonate, and the like. (See, for example, Berge S. M. et al., “Pharmaceutical Salts,” J. Pharm. Sci., 1977; 66:1-19 which is incorporated herein by reference.)

Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. Preferred esters include C₁-C₄ alkyl. Esters of the compounds of the present invention may be prepared according to conventional methods “March's Advanced Organic Chemistry, 5^{th} Edition”. M. B. Smith & J. March, John Wiley & Sons, 2001.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods such as “March's Advanced Organic Chemistry, 5^{th} Edition”. M. B. Smith & J. March, John Wiley & Sons, 2001.

The term “prodrug” refers to compounds that are rapidly transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drua Design, ed. Edward B. Roche, American Pharmaceutical Association and Pergamon Press, 1987, both of which are hereby incorporated by reference.

Preferred compounds of the present invention are those having the Formula IA:

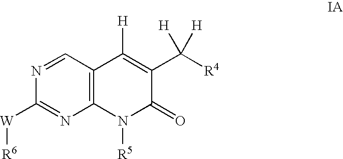

Embodiments of the present invention include, but are not limited to, the compounds listed below and their pharmaceutically acceptable salts:
(8-Cyclopentyl-2-methylsulfanyl-7-oxo-7,8-dihydro-pyrido[2,3-d]pyrimidin-6-yl)-acetic acid N′-acetyl-hydrazide;
8-Cyclopentyl-6-(5-methyl-[1,3,4]oxadiazol-2-ylmethyl)-2-methylsulfanyl-8H-pyrido[2,3-d]pyrimidin-7-one;
8-Cyclopentyl-2-methanesulfinyl-6-(5-methyl-[1,3,4]oxadiazol-2-ylmethyl)-8H-pyrido[2,3-d]pyrimidin-7-one;
4-{4-[8-Cyclopentyl-6-(5-methyl-[1,3,4]oxadiazol-2-ylmethyl)-7-oxo-7,8-dihydro-pyrido[2,3-d]pyrimidin-2-ylamino]-phenyl}-piperazine-1-carboxylic acid tert-butyl ester;
6-Benzyl-8-cyclopentyl-2-methylsulfanyl-8H-pyrido[2,3-d]pyrimidin-7-one;
6-Benzyl-8-cyclopentyl-2-methanesulfinyl-8H-pyrido[2,3-d]pyrimidin-7-one;
4-[4-(6-Benzyl-8-cyclopentyl-7-oxo-7,8-dihydro-pyrido[2,3-d]pyrimidin-2-ylamino)-phenyl]-piperazine-1-carboxylic acid tert-butyl ester;
8-Cyclopentyl-2-methylsulfanyl-6-(1-phenyl-ethyl)-8H-pyrido[2,3-d]pyrimidin-7-one;
8-Cyclopentyl-2-methanesulfinyl-6-(1-phenyl-ethyl)-8H-pyrido[2,3-d]pyrimidin-7-one;
4-{4-[8-Cyclopentyl-7-oxo-6-(1-phenyl-ethyl)-7,8-dihydro-pyrido[2,3-d]pyrimidin-2-ylamino]-phenyl}-piperazine-1-carboxylic acid tert-butyl ester;
8-Cyclopentyl-6-(1-phenyl-ethyl)-2-(4-piperazin-1-yl-phenylamino)-8H-pyrido[2,3-d]pyrimidin-7-one;
6-Benzoyl-8-cyclopentyl-2-methylsulfanyl-8H-pyrido[2,3-d]pyrimidin-7-one
6-Benzoyl-8-cyclopentyl-2-methanesulfinyl-8H-pyrido[2,3-d]pyrimidin-7-one;
4-[4-(6-Benzoyl-8-cyclopentyl-7-oxo-7,8-dihydro-pyrido[2,3-d]pyrimidin-2-ylamino)-phenyl]-piperazine-1-carboxylic acid tert-butyl ester;
8-Cyclopentyl-2-methylsulfanyl-6-thiazol-2-ylmethyl-8H-pyrido[2,3-d]pyrimidin-7-one;
8-Cyclopentyl-2-methanesulfinyl-6-thiazol-2-ylmethyl-8H-pyrido[2,3-d]pyrimidin-7-one;
4-[4-(8-Cyclopentyl-7-oxo-6-thiazol-2-ylmethyl-7,8-dihydro-pyrido[2,3-d]pyrimidin-2-ylamino)-phenyl]-piperazine-1-carboxylic acid tert-butyl ester;
Acetic acid N-[2-(8-cyclopentyl-2-methylsulfanyl-7-oxo-7,8-dihydro-pyrido[2,3-d]pyrimidin-6-yl)-acetyl]-hydrazide;
8-cyclopentyl-2-methylsulfanyl-6-pyridin-2-ylmethyl-8H-pyrido[2,3-d]pyrimidin-7-one;
8-Cyclopentyl-2-(1-methanesulfony-piperidin-4-ylamino)-6-pyridin-2-ylmethyl-8H-pyrido[2,3-d]pyrimidin-7-one;
8-Cyclopentyl-6-(hydroxy-phenyl-methyl)-5-methyl-2-methylsulfanyl-8H-pyrido[2,3-d]pyrimidin-7-one;
4-[4-(6-Benzoyl-8-cyclopentyl-5-methyl-7-oxo-7,8-dihydro-pyrido[2,3-d]pyrimidin-2-ylamino)-phenyl]-piperazine-1-carboxylic acid tert-butyl ester;
3-(4-Hydroxy-6-methyl-2-methylsulfanyl-pyrimidin-5-yl)-propionic acid ethyl ester;
3-(4-Chloro-6-methyl-2-methylsulfanyl-pyrimidin-5-yl)-propionic acid ethyl ester;
8-Cyclopentyl-4-methyl-2-methylsulfanyl-5,8-dihydro-6H-pyrido[2,3-d]pyrimidin-7-one;
6-Benzyl-8-cyclopentyl-4-methyl-2-methylsulfanyl-8H-pyrido[2,3-d]pyrimidin-7-one;
6-Benzyl-8-cyclopentyl-2-[1-(4-methyl-piperazine-1-sulfonyl)-piperidin-4-ylamino]-8H-pyrido[2,3-d]pyrimidin-7-one;
6-Benzyl-8-cyclopentyl-2-[1-(propane-2-sulfonyl)-piperidin-4-ylamino]-8H-pyrido[2,3-d]pyrimidin-7-one;
8-Cyclopentyl-2-(1-methanesulfonyl-piperidin-4-ylamino)-6-pyridin-2-ylmethyl-8H-pyrido[2,3-d]pyrimidin-7-one;
8-Cyclopentyl-2-[1-(propane-2-sulfonyl)-piperidin-4-ylamino]-6-pyridin-2-ylmethyl-8H-pyrido[2,3-d]pyrimidin-7-one;
4-[4-(8-Cyclopentyl-7-oxo-6-pyridin-2-ylmethyl-7,8-dihydro-pyrido[2,3-d]pyrimidin-2-ylamino)-phenyl]-piperazine-1-carboxylic acid tert-butyl ester;
6-Benzyl-2-benzylamino-8-cyclopentyl-8H-pyrido[2,3-d]pyrimidin-7-one;
6-Benzyl-8-cyclopentyl-2-phenylamino-8H-pyrido[2,3-d]pyrimidin-7-one;
6-Benzyl-8-cyclopentyl-2-[(2-hydroxy-ethyl)-methyl-amino]-8H-pyrido[2,3-d]pyrimidin-7-one;
6-Benzyl-8-cyclopentyl-2-(3-imidazol-1-yl-propylamino)-8H-pyrido[2,3-d]pyrimidin-7-one;
6-Benzyl-8-cyclopentyl-2-[2-(3H-imidazol-4-yl)-ethylamino]-8H-pyrido[2,3-d]pyrimidin-7-one;
3-(6-Benzyl-8-cyclopentyl-7-oxo-7,8-dihydro-pyrido[2,3-d]pyrimidin-2-ylamino)-propionic acid;
8-Cyclopentyl-6-(4-methoxy-benzyl)-2-(4-piperazin-1-yl-phenylamino)-8H-pyrido[2,3-d]pyrimidin-7-one;
8-Cyclopentyl-2-(4-piperazin-1-yl-phenylamino)-6-(4-trifluoromethyl-benzyl)-8H-pyrido[2,3-d]pyrimidin-7-one;
6-(4-Chloro-benzyl)-8-cyclopentyl-2-(4-piperazin-1 -yl-phenylamino)-8H-pyrido[2,3-d]pyrimidin-7-one;
8-Cyclopentyl-6-(4-methyl-benzyl)-2-(4-piperazin-1-yl-phenylamino)-8H-pyrido[2,3-d]pyrimidin-7-one;
8-Cyclopentyl-6-(4-methyl-benzyl)-2-(1-propyl-piperidin-4-ylamino)-8H-pyrido[2,3-d]pyrimidin-7-one;
8-Cyclopentyl-6-(2-methyl-benzyl)-2-(1-propyl-piperidin-4-ylamino)-8H-pyrido[2,3-d]pyrimidin-7-one;
8-Cyclopentyl-6-(3-methyl-benzyl)-2-(1-propyl-piperidin-4-ylamino)-8H-pyrido[2,3-d]pyrimidin-7-one;
8-Cyclopentyl-6-(4-ethyl-benzyl)-2-(1-propyl-piperidin-4-ylamino)-8H-pyrido[2,3-d]pyrimidin-7-one;
8-Cyclopentyl-6-(4-isopropyl-benzyl)-2-(1-propyl-piperidin-4-ylamino)-8H-pyrido[2,3-d]pyrimidin-7-one;
8-Cyclopentyl-2-(4-dimethylamino-cyclohexylamino)-6-(2-methyl-benzyl)-8H-pyrido[2,3-d]pyrimidin-7-one;
8-Cyclopentyl-2-(4-dimethylamino-cyclohexylamino)-6-(3-methyl-benzyl)-8H-pyrido[2,3-d]pyrimidin-7-one;
8-Cyclopentyl-2-(4-dimethylamino-cyclohexylamino)-6-(4-methyl-benzyl)-8H-pyrido[2,3-d]pyrimidin-7-one;
8-Cyclopentyl-2-(4-dimethylamino-cyclohexlyamino)-6-(4-ethyl-benzyl)-8H-pyrido[2,3-d]pyrimidin-7-one;
8-Cyclopentyl-2-(4-dimethylamino-cyclohexylamino)-6-(4-isopropyl-benzyl)-8H-pyrido[2,3-d]pyrimidin-7-one;
8-Cyclopentyl-6-(2-methyl-benzyl)-2-(4-piperazin-1-yl-phenylamino)-8H-pyrido[2,3-d]pyrimidin-7-one;
8-Cyclopentyl-6-(3-methyl-benzyl)-2-(4-piperazin-1-yl-phenylamino)-8H-pyrido[2,3-d]pyrimidin-7-one;
8-Cyclopentyl-6-(4-ethyl-benzyl)-2-(4-piperazin-1-yl-phenylamino)-8H-pyrido[2,3-d]pyrimidin-7-one;
8-Cyclopentyl-6-(4-isopropyl-benzyl)-2-(4-piperazin-1-yl-phenylamino)-8H-pyrido[2,3-d]pyrimidin-7-one;
8-Cyclopentyl-6-(2-methyl-benzyl)-2-(2-morpholin-4-yl-ethylamino)-8H-pyrido[2,3-d]pyrimidin-7-one;
8-Cyclopentyl-6-(2-methyl-benzyl)-2-(3-morpholin-4-yl-propylamino)-8H-pyrido[2,3-d]pyrimidin-7-one;
8-Cyclopentyl-6-(4-methyl-benzyl)-2-(2-morpholin-4-yl-ethylamino)-8H-pyrido[2,3-d]pyrimidin-7-one;
8-Cyclopentyl-6-(4-methyl-benzyl)-2-(3-morpholin-4-yl-propylamino)-8H-pyrido[2,3-d]pyrimidin-7-one;
8-Cyclopentyl-6-(2-methyl-benzyl)-2-[3-(4-methyl-piperazin-1-yl)-propylamino]-8H-pyrido[2,3-d]pyrimidin-7-one;
8-Cyclopentyl-6-(4-methyl-benzyl)-2-[3-(4-methyl-piperazin-1-yl)-propylamino]-8H-pyrido[2,3-d]pyrimidin-7-one;
6-Benzyl-8-cyclopentyl-2-(2-dimethylamino-ethylamino)-8H-pyrido[2,3-d]pyrimidin-7-one;
6-Benzyl-8-cyclopentyl-2-(3-dimethylamino-propylamino)-8H-pyrido[2,3-d]pyrimidin-7-one;
8-Cyclopentyl-2-(4-dimethylamino-butylamino)-6-(2-methyl-benzyl)-8H-pyrido[2,3-d]pyrimidin-7-one;
8-Cyclopentyl-2-(3-dimethylamino-propylamino)-6-(2-methyl-benzyl)-8H-pyrido[2,3-d]pyrimidin-7-one;
8-Cyclopentyl-2-(3-dimethylamino-propylamino)-6-(4-methyl-benzyl)-8H-pyrido[2,3-d]pyrimidin-7-one;
8-Cyclopentyl-2-(4-dimethylamino-cyclohexylamino)-6-(4-methyl-benzyl)-8H-pyrido[2,3-d]pyrimidin-7-one;
8-Cyclopentyl-2-(4-dimethylamino-cyclohexylamino)-6-(4-methyl-benzyl)-8H-pyrido[2,3-d]pyrimidin-7-one;
8-Cyclopentyl-2-(1-propyl-piperidin-4-ylamino)-6-thiazol-2-ylmethyl-8H-pyrido[2,3-d]pyrimidin-7-one;
8-Cyclopentyl-2-(4-dimethylamino-butylamino)-6-pyridin-2-ylmethyl-8H-pyrido[2,3-d]pyrimidin-7-one;
8-Cyclopentyl-2-(4-dimethylamino-butylamino)-6-thiazol-2-ylmethyl-8H-pyrido[2,3-d]pyrimidin-7-one;
8-Cyclopentyl-2-[3-(4-methyl-piperazin-1-yl)-propylamino]-6-thiazol-2-ylmethyl-8H-pyrido[2,3-d]pyrimidin-7-one;
8-Cyclopentyl-2-[4-(4-methyl-piperazin-1-yl)-butylamino]-6-thiazol-2-ylmethyl-8H-pyrido[2,3-d]pyrimidin-7-one;
8-Cyclopentyl-6-(3-methyl-benzyl)-2-(piperidin-4-ylamino)-8H-pyrido[2,3-d]pyrimidin-7-one;
8-Cyclopentyl-6-(4-methyl-benzyl)-2-(piperidin-4-ylamino)-8H-pyrido[2,3-d]pyrimidin-7-one;
8-Cyclopentyl-2-[5-(4-methyl-piperazin-1-yl)-pentylamino]-6-thiazol-2-ylmethyl-8H-pyrido[2,3-d]pyrimidin-7-one;
8-Cyclopentyl-2-(5-morpholin-4-yl-pentylamino)-6-thiazol-2-ylmethyl-8H-pyrido[2,3-d]pyrimidin-7-one;
8-Cyclopentyl-2-(5-diisopropylamino-pentylamino)-6-thiazol-2-ylmethyl-8H-pyrido[2,3-d]pyrimidin-7-one;
8-Cyclopentyl-2-(3-diethylamino-propylamino)-6-thiazol-2-ylmethyl-8H-pyrido[2,3-d]pyrimidin-7-one;
8-Cyclopentyl-2-(6-dimethylamino-hexylamino)-6-thiazol-2-ylmethyl-8H-pyrido[2,3-d]pyrimidin-7-one;
8-Cyclopentyl-6-(5-methyl-[1,3,4]oxadiazol-2-ylmethyl)-2-[4-(4-methyl-piperazin-1-yl)-butylamino]-8H-pyrido[2,3-d]pyrimidin-7-one;
8-Cyclopentyl-6-(5-methyl-[1,3,4]oxadiazol-2-ylmethyl)-2-[5-(4-methyl-piperazin-1-yl)-pentylamino]-8H-pyrido[2,3-d]pyrimidin-7-one;
8-Cyclopentyl-6-(5-methyl-[1,3,4]oxadiazol-2-ylmethyl)-2-(piperidin-4-ylamino)-8H-pyrido[2,3-d]pyrimidin-7-one;
8-Cyclopentyl-6-(4-methyl-benzyl)-2-[4-(2-oxo-oxazolidin-3-yl)-phenylamino]-8H-pyrido[2,3-d]pyrimidin-7-one;
6-Benzyl-8-cyclopentyl-2-(2-hydroxy-ethylamino)-8H-pyrido[2,3-d]pyrimidin-7-one;
6-Benzyl-8-cyclopentyl-2-(2-methoxy-ethylamino)-8H-pyrido[2,3-d]pyrimidin-7-one;
6-Benzyl-8-cyclopentyl-2-(2-morpholin-4-yl-ethylamino)-8H-pyrido[2,3-d]pyrimidin-7-one;
6-Benzyl-8-cyclopentyl-2-(3-methyl-butylamino)-8H-pyrido[2,3-d]pyrimidin-7-one;
6-Benzyl-8-cyclopentyl-2-[2-(2-hydroxy-ethoxy)-ethylamino]-8H-pyrido[2,3-d]pyrimidin-7-one;
6-Benzyl-8-cyclopentyl-2-(2-diethylamino-ethylamino)-8H-pyrido[2,3-d]pyrimidin-7-one;
6-Benzyl-8-cyclopentyl-2-(2-hydroxy-1-methyl-ethylamino)-8H-pyrido[2,3-d]pyrimidin-7-one;
6-Benzyl-8-cyclopentyl-2-isopropylamino-8H-pyrido[2,3-d]pyrimidin-7-one;
2-(2-Amino-ethylamino)-6-benzyl-8-cyclopentyl-8H-pyrido[2,3-d]pyrimidin-7-one;
6-Benzyl-8-cyclopentyl-2-cyclopentylamino-8H-pyrido[2,3-d]pyrimidin-7-one;
6-Benzyl-8-cyclopentyl-2-(2,3-dihydroxy-propylamino)-8H-pyrido[2,3-d]pyrimidin-7-one;
6-Benzyl-8-cyclopentyl-2-(1,3-dimethyl-butylamino)-8H-pyrido[2,3-d]pyrimidin-7-one;
6-Benzyl-8-cyclopentyl-2-(2-dimethylamino-1-methyl-ethylamino)-8H-pyrido[2,3-d]pyrimidin-7-one;
6-Benzyl-8-cyclopentyl-2-(1-ethynyl-cyclohexylamino)-8H-pyrido[2,3-d]pyrimidin-7-one;
6-Benzyl-8-cyclopentyl-2-(3-pyrrolidin-1-yl-propylamino)-8H-pyrido[2,3-d]pyrimidin-7-one;
6-Benzyl-8-cyclopentyl-2-(3-morpholin-4-yl-propylamino)-8H-pyrido[2,3-d]pyrimidin-7-one;
6-Benzyl-8-cyclopentyl-2-(2-methoxy-1-methyl-ethylamino)-8H-pyrido[2,3-d]pyrimidin-7-one;
6-Benzyl-8-cyclopentyl-2-(3-methoxy-propylamino)-8H-pyrido[2,3-d]pyrimidin-7-one;
[4-(6-Benzyl-8-cyclopentyl-7-oxo-7,8-dihydro-pyrido[2,3-d]pyrimidin-2-ylamino)-cyclohexyl]-acetic acid;
6-Benzyl-8-cyclopentyl-2-(4-hydroxy-cyclohexylamino)-8H-pyrido[2,3-d]pyrimidin-7-one;
6-Benzyl-8-cyclopentyl-2-(1-ethyl-piperidin-3-ylamino)-8H-pyrido[2,3-d]pyrimidin-7-one;
8-Cyclopentyl-2-(2-methoxy-ethylamino)-6-(1-phenyl-ethyl)-8H-pyrido[2,3-d]pyrimidin-7-one;
8-Cyclopentyl-2-(3-methyl-butylamino)-6-(1-phenyl-ethyl)-8H-pyrido[2,3-d]pyrimidin-7-one;
8-Cyclopentyl-2-[2-(2-hydroxy-ethoxy)-ethylamino]-6-(1-phenyl-ethyl)-8H-pyrido[2,3-d]pyrimidin-7-one;
8-Cyclopentyl-2-(2-diethylamino-ethylamino)-6-(1-phenyl-ethyl)-8H-pyrido[2,3-d]pyrimidin-7-one;
8-Cyclopentyl-2-isopropylamino-6-(1-phenyl-ethyl)-8H-pyrido[2,3-d]pyrimidin-7-one;
8-Cyclopentyl-2-(2-hydroxy-1-methyl-ethylamino)-6-(1-phenyl-ethyl)-8H-pyrido[2,3-d]pyrimidin-7-one;
8-Cyclopentyl-2-(2-morpholin-4-yl-ethylamino)-6-(1-phenyl-ethyl)-8H-pyrido[2,3-d]pyrimidin-7-one;
2-(2-Amino-ethylamino)-8-cyclopentyl-6-(1-phenyl-ethyl)-8H-pyrido[2,3-d]pyrimidin-7-one;
8-Cyclopentyl-2-cyclopentylamino-6-(1-phenyl-ethyl)-8H-pyrido[2,3-d]pyrimidin-7-one;
8-Cyclopentyl-2-(1,3-dimethyl-butylamino)-6-(1-phenyl-ethyl)-8H-pyrido[2,3-d]pyrimidin-7-one;
8-Cyclopentyl-2-(3,3-dimethyl-butylamino)-6-(1-phenyl-ethyl)-8H-pyrido[2,3-d]pyrimidin-7-one;
8-Cyclopentyl-2-(2-dimethylamino-1-methyl-ethylamino)-6-(1-phenyl-ethyl)-8H-pyrido[2,3-d]pyrimidin-7-one;
8-Cyclopentyl-2-(3-methylsulfanyl-propylamino)-6-(1-phenyl-ethyl)-8H-pyrido[2,3-d]pyrimidin-7-one;
8-Cyclopentyl-2-(1,4-dimethyl-pentylamino)-6-(1-phenyl-ethyl)-8H-pyrido[2,3-d]pyrimidin-7-one;
8-Cyclopentyl-2-(1-ethynyl-cyclohexylamino)-6-(1-phenyl-ethyl)-8H-pyrido[2,3-d]pyrimidin-7-one;
8-Cyclopentyl-2-(3-morpholin-4-yl-propylamino)-6-(1-phenyl-ethyl)-8H-pyrido[2,3-d]pyrimidin-7-one;
8-Cyclopentyl-2-(2-methoxy-1-methyl-ethylamino)-6-(1-phenyl-ethyl)-8H-pyrido[2,3-d]pyrimidin-7-one;
8-Cyclopentyl-2-(2-hydroxy-ethylamino)-6-(1-phenyl-ethyl)-8H-pyrido[2,3-d]pyrimidin-7-one;
2-Cyclohexylamino-8-cyclopentyl-6-(1-phenyl-ethyl)-8H-pyrido[2,3-d]pyrimidin-7-one;
2-(4-Amino-cyclohexylamino)-8-cyclopentyl-6-(1-phenyl-ethyl)-8H-pyrido[2,3-d]pyrimidin-7-one;
8-Cyclopentyl-2-(3-methoxy-propylamino)-6-(1-phenyl-ethyl)-8H-pyrido[2,3-d]pyrimidin-7-one
8-Cyclopentyl-2-(4-methyl-cyclohexylamino)-6-(1-phenyl-ethyl)-8H-pyrido[2,3-d]pyrimidin-7-one;
4-[8-Cyclopentyl-7-oxo-6-(1-phenyl-ethyl)-7,8-dihydro-pyrido[2,3-d]pyrimidin-2-ylamino]-cyclohexanecarboxylic acid;
8-Cyclopentyl-2-(4-diethylamino-cyclohexylamino)-6-(1-phenyl-ethyl)-8H-pyrido[2,3-d]pyrimidin-7-one;
{4-[8-Cyclopentyl-7-oxo-6-(1-phenyl-ethyl)-7,8-dihydro-pyrido[2,3-d]pyrimidin-2-ylamino]-cyclohexyl}-acetic acid;
8-Cyclopentyl-2-(4-hydroxy-cyclohexylamino)-6-(1-phenyl-ethyl)-8H-pyrido[2,3-d]pyrimidin-7-one;
8-Cyclopentyl-2-(1-ethyl-piperidin-3-ylamino)-6-(1-phenyl-ethyl)-8H-pyrido[2,3-d]pyrimidin-7-one;
8-Cyclopentyl-6-(1-phenyl-ethyl)-2-(1-propyl-piperidin-4-ylamino)-8H-pyrido[2,3-d]pyrimidin-7-one;
8-Cyclopentyl-6-(1-phenyl-ethyl)-2-(3-piperidin-1-yl-propylamino)-8H-pyrido[2,3-d]pyrimidin-7one; and
8-Cyclopentyl-2-(4-dimethylamino-cyclohexylamino)-6-(1-phenyl-ethyl)-8H-pyrido[2,3-d]pyrimidin-7-one;

Other embodiments include:
8-Cyclopentyl-6-(5-methyl-oxazol-2-ylmethyl)-2-(4-piperazin-1-yl-phenylamino)-8H-pyrido[2,3-d]pyrimidin-7-one;
8-Cyclopentyl-6-(4-methyl-oxazol-2-ylmethyl)-2-(4-piperazin-1-yl-phenylamino)-8H-pyrido[2,3-d]pyrimidin-7-one;
8-Cyclopentyl-6-(2-methyl-oxazol-4-ylmethyl)-2-(4-piperazin-1-yl-phenylamino)-8H-pyrido[2,3-d]pyrimidin-7-one;
8-Cyclopentyl-6-isoxazol-4-ylmethyl-2-(4-piperazin-1-yl-phenylamino)-8H-pyrido[2,3-d]pyrimidin-7-one;
8-Cyclopentyl-6-isoxazol-5-ylmethyl-2-(4-piperazin-1-yl-phenylamino)-8H-pyrido[2,3-d]pyrimidin-7-one;
8-Cyclopentyl-6-isoxazol-3-ylmethyl-2-(4-piperazin-1-yl-phenylamino)-8H-pyrido[2,3-d]pyrimidin-7-one;
8-Cyclopentyl-6-(5-methyl-oxazol-2-ylmethyl)-2-(1-propyl-piperidin-4-ylamino)-8H-pyrido[2,3-d]pyrimidin-7-one;
8-Cyclopentyl-2-(4-dimethylamino-cyclohexylamino)-6-(5-methyl-oxazol-2-ylmethyl)-8H-pyrido[2,3-d]pyrimidin-7-one;
8-Cyclopentyl-6-(5-methyl-oxazol-2-ylmethyl)-2-(piperidin-4-ylamino)-8H-pyrido[2,3-d]pyrimidin-7-one;
8-Cyclopentyl-2-(4-piperazin-1-yl-phenylamino)-6-(1H-pyrrol-2-ylmethyl)-8H-pyrido[2,3-d]pyrimidin-7-one;
8-Isopropyl-2-(5-piperazin-1-yl-pyridin-2-ylamino)-6-(1H-pyrrol-2-ylmethyl)-8H-pyrido[2,3-d]pyrimidin-7-one
2-(4-Dimethylamino-cyclohexylamino)-8-ethyl-6-(1H-pyrrol-2-ylmethyl)-8H-pyrido[2,3-d]pyrimidin-7-one;
8-Isopropyl-6-(5-methyl-furan-2-ylmethyl)-2-(4-piperazin-1-yl-phenylamino)-8H-pyrido[2,3-d]pyrimidin-7-one;
8-Ethyl-6-(5-methyl-furan-2-ylmethyl)-2-(5-piperazin-1-yl-pyridin-2-ylamino)-8H-pyrido[2,3-d]pyrimidin-7-one;
8-Cyclopentyl-2-(4-dimethylamino-cyclohexylamino)-6-(5-methyl-furan-2-ylmethyl)-8H-pyrido[2,3-d]pyrimidin-7-one;
6-(1H-Imidazol-2-ylmethyl)-2-(4-piperazin-1-yl-phenylamino)-8-(tetrahydro-furan-3-ylmethyl)-8H-pyrido[2,3-d]pyrimidin-7-one;
2-[8-(2-Methoxy-ethyl)-7-oxo-2-(5-piperazin-1-yl-pyridin-2-ylamino)-7,8-dihydro-pyridino[2,3-d]pyrimidin-6-ylmethyl]-1H-imidazole-4-carboxylic acid ethyl ester;
2-(4-Dimethylamino-cyclohexylamino)-6-(1H-imidazol-2-ylmethyl)-8-(tetrahydro-pyran-4-yl)-8H-pyrido[2,3-d]pyrimidin-7-one;
8-(2-Cyclopropyl-ethyl)-6-(1H-indol-2-ylmethyl)-2-(4-piperazin-1-yl-phenylamino)-8H-pyrido[2,3-d]pyrimidin-7-one;
6-Benzooxazol-6-ylmethyl-8-(2-methoxy-ethyl)-2-(5-piperazin-1-yl-pyridin-2-ylamino)-8H-pyrido[2,3-d]pyrimidin-7-one; and
6-Benzofuran-6-ylmethyl-2-(4-dimethylamino-cyclohexylamino)-8-(tetrahydro-pyran-4-yl)-8H-pyrido[2,3-d]pyrimidin-7-one.

The most preferred compounds of the present invention are:
8-Cyclopentyl-6-(5-methyl-[1,3,4]oxadiazol-2-ylmethyl)-2-(4-piperazin-1-yl-phenylamino)-8H-pyrido[2,3-d]pyrimidin-7-one;
6-Benzyl-8-cyclopentyl-2-(4-piperazin-1-yl-phenylamino)-8H-pyrido[2,3-d]pyrimidin-7-one;
6-Benzoyl-8-cyclopentyl-2-(4-piperazin-1-yl-phenylamino)-8H-pyrido[2,3-d]pyrimidin-7-one;
8-Cyclopentyl-2-(4-piperazin-1-yl-phenylamino)-6-thiazol-2-ylmethyl-8H-pyrido[2,3-d]pyrimidin-7-one;
8-Cyclopentyl-6-(5-methyl-[1,3,4]oxadiazol-2-ylmethyl)-2-methylsulfanyl-8H-pyrido[2,3-d]pyrimidin-7-one;
8-Cyclopentyl-2-(piperidin-4-ylamino)-6-pyridin-2-ylmethyl-8H-pyrido[2,3-d]pyrimidin-7-one;
6-Benzyl-8-cyclopentyl-5-methyl-2-(4-piperazin-1-yl-phenylamino)-8H-pyrido[2,3-d]pyrimidin-7-one;
8-Cyclopentyl-6-(hydroxy-phenyl-methyl)-5-methyl-2-(4-piperazin-1-yl-phenylamino)-8H-pyrido[2,3-d]pyrimidin-7-one;
6-Benzoyl-8-cyclopentyl-5-methyl-2-(4-piperazin-1-yl-phenylamino)-8H-pyrido[2,3-d]pyrimidin-7-one;
8-Cyclopentyl-2-(4-piperazin-1-yl-phenylamino)-6-pyridin-2-ylmethyl-8H-pyrido[2,3-d]pyrimidin-7-one;
8-Cyclopentyl-2-phenylamino-6-pyridin-2-ylmethyl-8H-pyrido[2,3-d]pyrimidin-7-one;
8-Cyclopentyl-6-(3,4-dimethoxy-benzyl)-2-(4-piperazin-1-yl-phenylamino)-8H-pyrido[2,3-d]pyrimidin-7-one;
8-Cyclopentyl-2-[4-(2-oxo-oxazolidin-3-yl)-phenylamino]-6-(1-phenyl-ethyl)-8H-pyrido[2,3-d]pyrimidin-7-one;
6-Benzyl-8-cyclopentyl-2-(4-dimethylamino-cyclohexylamino)-8H-pyrido[2,3-d]pyrimidin-7-one;
6-Benzyl-2-cyclohexylamino-8-cyclopentyl-8H-pyrido[2,3-d]pyrimidin-7-one;
6-Benzyl-8-cyclopentyl-2-(2-hydroxy-ethylamino)-8H-pyrido[2,3-d]pyrimidin-7-one;
6-Benzyl-8-cyclopentyl-2-(1-propyl-piperidin-4-ylamino)-8H-pyrido[2,3-d]pyrimidin-7-one;
6-Benzyl-8-cyclopentyl-2-(piperidin-4-ylamino)-8H-pyrido[2,3-d]pyrimidin-7-one;
6-Benzyl-8-cyclopentyl-2-methylamino-8H-pyrido[2,3-d]pyrimidin-7-one;
6-Benzyl-8-cyclopentyl-2-dimethylamino-8H-pyrido[2,3-d]pyrimidin-7-one;
6-Benzyl-8-cyclopentyl-2-(5-piperazin-1-yl-pyridin-2-ylamino)-8H-pyrido[2,3-d]pyrimidin-7-one;
6-Benzyl-8-cyclopentyl-2-(1-methanesulfonyl-piperidin-4-ylamino)-8H-pyrido[2,3-d]pyrimidin-7-one;
8-Cyclopentyl-2-(4-dimethylamino-cyclohexylamino)-6-thiazol-2-ylmethyl-8H-pyrido[2,3-d]pyrimidin-7-one; and
8-Cyclopentyl-2-(1-propyl-piperidin-4-ylamino)-6-pyridin-2-ylmethyl-8H-pyrido[2,3-d]pyrimidin-7-one.

This invention provides a method of treating a disorder or condition selected from the group consisting of cell proliferative disorders, such as cancer, vascular smooth muscle proliferation associated with atherosclerosis, postsurgical vascular stenosis, restenosis, and endometriosis; infections, including viral infections such as DNA viruses like herpes and RNA viruses like HIV, and fungal infections; autoimmune diseases such as psoriasis, inflammation like rheumatoid arthritis, lupus, type 1 diabetes, diabetic nephropathy, multiple sclerosis, and glomerulonephritis, organ transplant rejection, including host versus graft disease, in a mammal, including human, comprising administering to said mammal an amount of a compound of formula I, or a pharmaceutically acceptable salt thereof, that is effective in treating such disorder or condition

This invention further provides compounds of formula I that are useful for treating abnormal cell proliferation such a cancer. The invention provides a method of treating the abnormal cell proliferation disorders such as a cancer selected from the group consisting of cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia, comprising administering a therapeutically effective amount of a compound of formula I, or a pharmaceutically acceptable salt thereof, to a subject in need of such treatment.

A further embodiment of this invention is a method of treating subjects suffering from diseases caused by vascular smooth muscle cell proliferation. Compounds within the scope of the present invention effectively inhibit vascular smooth muscle cell proliferation and migration. The method comprises administering to a subject in need of treatment an amount of a compound of formula I, or a pharmaceutically acceptable salt thereof, sufficient to inhibit vascular smooth muscle proliferation, and/or migration.

This invention further provides a method of treating a subject suffering from gout comprising administering to said subject an amount of a compound of formula I, or a pharmaceutically acceptable salt thereof, sufficient to treat the condition.

This invention further provides a method of treating a subject suffering from kidney disease, such as polycystic kidney disease, comprising administering to said subject in need of treatment an amount of a compound of formula I, or a pharmaceutically acceptable salt thereof, sufficient to treat the condition.

Because of the selective inhibitory activity against Cdks and other kinases, the compounds of the present invention are also useful research tools for studying the mechanism of action of those kinases, both in vitro and in vivo.

The above-identified methods of treatment are preferably carried out by administering a therapeutically effective amount of a compound of Formula I to a subject in need of treatment.

Many of the compounds of the present invention are selective inhibitors of cyclin dependent kinase Cdk4, which is to say that they inhibit Cdk4 more potently than they inhibit tyrosine kinases and other serine-threonine kinases including other cyclin-dependent kinases such as Cdk2. Despite their selectivity for Cdk4 inhibition, compounds of the invention may inhibit other kinases, albeit at higher concentrations than those at which they inhibit Cdk4. However, compounds of the present invention also may inhibit Cdk6 at similar concentrations to those necessary for inhibition of Cdk4 since Cdk6 is structurally similar to and performs similar functions to Cdk4.

Preferred embodiments of the present invention are compounds of the formula I that inhibit Cdk4 at least about 10-fold more potently than they inhibit Cdk2.

A preferred embodiment of the present invention provides a method of inhibiting Cdk4 comprising administration of a preferred compound of Formula I in an amount that selectively inhibits Cdk4 over Cdk2. The term “selectively inhibits” means that the preferred compound inhibits Cdk4 at a lower dose than is required to inhibit Cdk2.

### DETAILED DESCRIPTION OF THE INVENTION

In the discussion that follows W, R¹, R², R³ R⁴, R⁵, R⁶, R⁷, R⁸, R⁹ and R¹⁰, m, n, T, and Q are defined as they are defined above in the Summary of the Invention. All terms used herein have their normal meaning unless defined otherwise in the specification.

Synthesis

Compounds of the present invention may be prepared using methods largely based upon procedures and approaches previously disclosed (WO 98/33798, WO 01/70741, U.S. patent application Ser. No. 10/345,778 and Barvian et al, J. Med. Chem. 2000, 43, 4606-4616). Preparation of the [2,3-d]pyrido-pyrimidines substituted at the carbon at the critical 6 position of the the [2,3-d]pyrido-pyrimidine is most readily achieved via Aldol chemistry in which aldehyde 1 is combined with the anion of a suitable ester 2 and the subsequent aldol product is dehydrated under standard conditions such as by heating in mild acid, or, if necessary, by application of a dehydrating reagent such as Burgess salt (Scheme 1). Oxidation of the methyl sulfide substituent on the carbon at the 2 position to a sulfoxide or sulfone, followed by displacement with an amine as described previously (e.g. Barvian et al, J. Med. Chem. 2000, 43, 4606-4616) allows for introduction of the amine side chain on the carbon at the 2 position (Scheme 2). In certain cases, it may be more feasible to modify the substituent on the carbon at the 6 position of the [2,3-d]pyrido-pyrimidine ring subsequent to introduction of the side chain on the carbon at the 2 position. In this case, the substituent on the carbon at the 6 position of the [2,3-d]pyrido-pyrimidine ring installed via the Aldol procedure may be a simple acid, carboxylic ester or related functional group which can be subsequently modified under standard procedures to produce heterocyclic groups such as oxadiazoles, thiazoles and the like (Scheme 3).

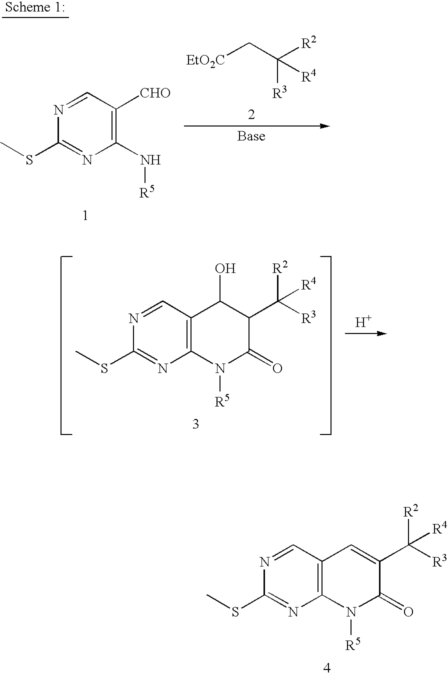

Scheme 1 demonstrates the preparation of the [2,3-d]pyrido-pyrimidine substituted at the carbon at the critical 6 position of the [2,3-d]pyrido-pyrimidine ring via Aldol chemistry in which aldehyde 1 is combined with the anion of a suitable ester 2 and the subsequent aldol product is dehydrated under standard conditions such as by heating in mild acid, or if necessary by application of a dehydrating reagent such as Burgess salt. Ester 2 is dissolved in an ethereal organic solvent such as THF or diethyl ether and cooled to a temperature between −100 and −50° C. The resulting product is then treated with an organic base such as LDA or LHMDS to deprotonate it at the carbon adjacent to the ester group. The resulting anion is added to the aldehyde 1 dissolved in an aprotic organic solvent at low temperature (−100 to −50° C.) and stirred for 10 minutes to 2 hours until all of the aldehyde has reacted. Suitable aprotic organic solvents may be selected from the group consisting of Tetrahydrofuran, Diethyl ether, Diglyme, Benzene, Toluene, Chlorobenzene, Dichloromethane, Dichloroethane, Chloroform, Hexanes, Pentane, Cyclohexane, Petroleum Ethers, and N-methylpyrrolidinone. The resulting mixture is allowed to warm slowly to room temperature over 1 to 5 hours. Usually the aldol product 3 spontaneously dehydrates under these conditions to produce the pyridopyrimidine product 4. In the event that this dehydration does not occur spontaneously, acidification of the reaction mixture with acetic acid or a sulfonic acid such a para-toluene sulfonic acid followed by gentle warming to 50-80° C. is generally sufficient to convert compound 3 to product 4. Other suitable acidifying reagents include, but are not limited to, Acetic Acid, para-Toluene sulfonic acid, Amberlyst, Hydrochloric acid, Sulfuric acid, Trifluoroacetic acid, and Methane sulfonic acid. In the event that compound 3 still fails to undergo dehydration, it is isolated and purified by standard procedures then dissolved in an organic solvent such as methylene chloride and treated with one equivalent of a dehydrating reagent such as Burgess's salt at room temperature until none of compound 3 remains. Another suitable dehydrating agent is Martin's Sulfurane. Those skilled in the art will be able to select other suitable reagents to carry out the exemplified reactions without undue experimentation.

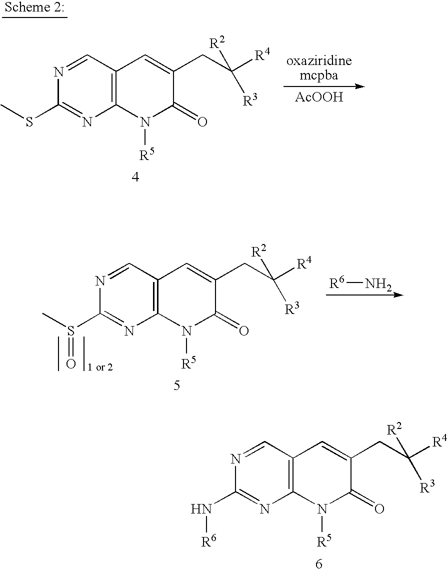

Oxidation of the methyl sulfide on the carbon at the 2 position of the pyrido[2,3d]pyrimidin ring produced in Scheme 1 to a sulfoxide or sulfone, followed by displacement with an amine, allows for introduction of the amine side chain on the carbon at the 2 position as exemplified in Scheme 2. Oxidation of sulfide 4 is performed using one of a variety of oxidants including, but not limited to, oxone, sodium meta-periodiate, meta-chloroperbenzoic acid, peracetic acid, hydrogen peroxide, sodium hypochlorite, 2-benzenesulfonyl-3-phenyl-oxaziridine depending upon the desired product. Thus, treatment of a solution of 4 in organic solvent such as methylene chloride or chloroform with one equivalent of an oxaziridine provides the sulfoxide cleanly and in good yield. Alternatively, treatment of the same solution of 4 instead with three equivalents of meta-chloroperbenzoic acid produces the sulfone. Displacement of either the sulfoxide or the sulfone (5) with an amine occurs upon heating to a temperature of 80-160° C. (step 5) with an excess of the amine (2-5 equivalents) in an aprotic organic solvent such as DMSO, toluene, acetonitrile, or xylene.

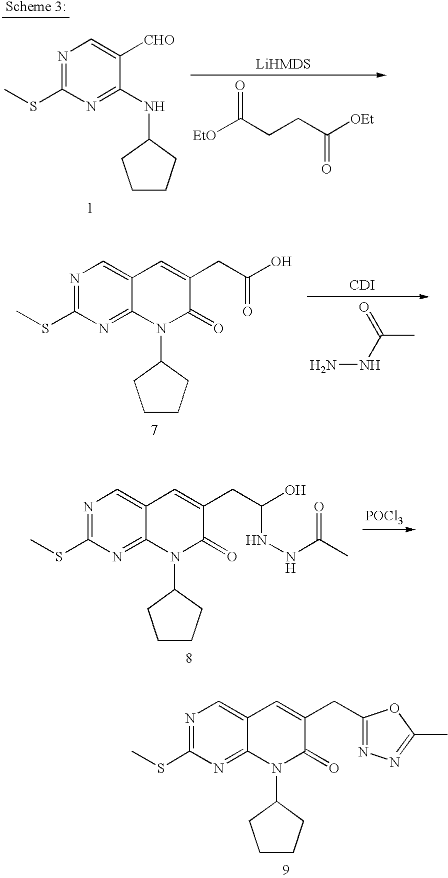

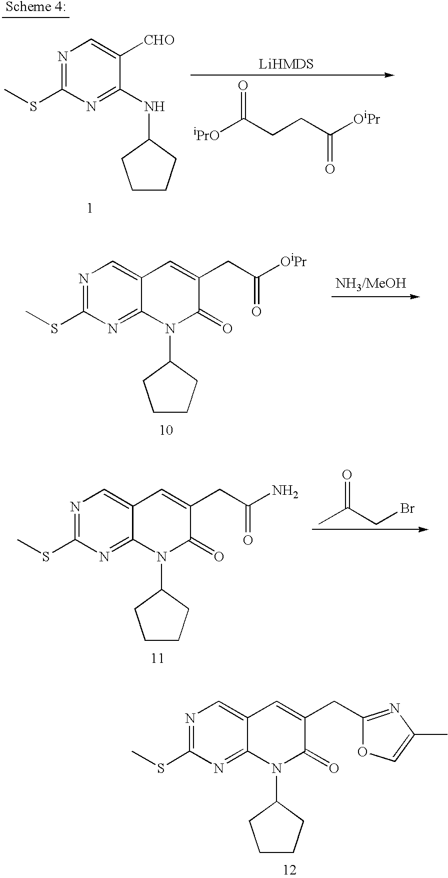

Schemes 3 and 4 depict alternate schemes wherein the substituent on the carbon at the 6 position of the pyrido[2,3d]pyrimidine ring is further modified prior to introduction of the side chain on the carbon at the 2 position of the pyrido[2,3d]pyrimidine ring. In this case, the substituent installed on the carbon at the 6 position of the pyrido[2,3d]pyrimidine ring via the Aldol procedure may be a simple acid, carboxylic ester or related functional group which can be subsequently modified under standard procedures to produce heteroaryl groups such as oxadiazoles, thiazoles and the like. In some cases carboxylic esters hydrolyse under the reaction conditions employed during the first step providing the ester directly as shown in Scheme 3. For example, diethyl succinate is dissolved in an aprotic organic solvent such as THF and cooled to −100 to −50° C. and deprotonated with a strong organic base such as LiHMDS for 2 to 20 minutes. The aldehyde (1) is added and after a short period of time (10 to 30 minutes) the reaction mixture is allowed to warm to room temperature. Following stirring at ambient temperature for 10 to 18 hours, the product is isolated by standard aqueous work up with acidification of the aqueous layer. Conversion of acid 7 to acyl hydrazide 8 is performed by dissolution of the acid in an organic solvent such as THF or dichloromethane, at room temperature, and addition of carbonyidiimidazole or a similar activating agent. After the acid group is activated (approximately 2 hours), acetohydrazine is added and stirring is continued for 10 to 15 hours at room temperature. The product is collected by filtration. The oxadiazole 9 is formed by treatment of the acylhydrazide 8 with POCl₃ (as solvent) at elevated temperatures (80-120° C.) for a period of time approximately 5 hours.

In other cases, with careful control of the reaction conditions, an ester is obtained from the first step (e.g. compound 10), which may be further modified prior to side chain installation at the 2-position of the pyrimidine ring, as in Scheme 4. Alternatively, the ester function on the carbon at the 6 position of the pyrido[2,3d]pyrimidin ring may be further modified subsequent to side chain installation at the 2-position of the pyrimidine ring, as in Schemes 5 and 6. In Scheme 5, oxidation of the C2 sulfide on the carbon at the 2 position of the ring 12, followed by heating with an amine in a nonpolar organic solvent to 80-160° C., provides compounds such as 14. Direct conversion of the ester to a primary amide by dissolution in an organic solvent such as THF or acetonitrile, treatment with ammonia gas under slightly elevated pressure (1.5-10 atmospheres) and temperature (50-100° C.) produces compound 15. Alternatively, the ester 14 maybe hydrolyzed by treatment with a mixture or aqueous sodium hydroxide (0.5-1 M) and an organic solvent (THF or acetonitrile) at 25 to 80° C. for 2 to 24 hours. Then, the resulting acid may be converted to the primary amide by activation with a coupling agent such as oxalyl chloride or carbonyidiimidazole and treatment with ammonia. Amide 15 may be converted to a variety of heteroaryl groups under standard conditions. For example, treatment of 15 with bromoacetone in a polar organic solvent with heating to 80-120° C. produces oxazoles as represented by structure 17.

Another good starting point for the construction of aromatic heterocycles is a terminal nitrile such as in compounds 18 and 20. Conversion of nitriles to oxazoles or thiazoles is achieved by heating the nitrile with an amino alcohol or amino thiol in a polar organic solvent such as ethanol at reflux.

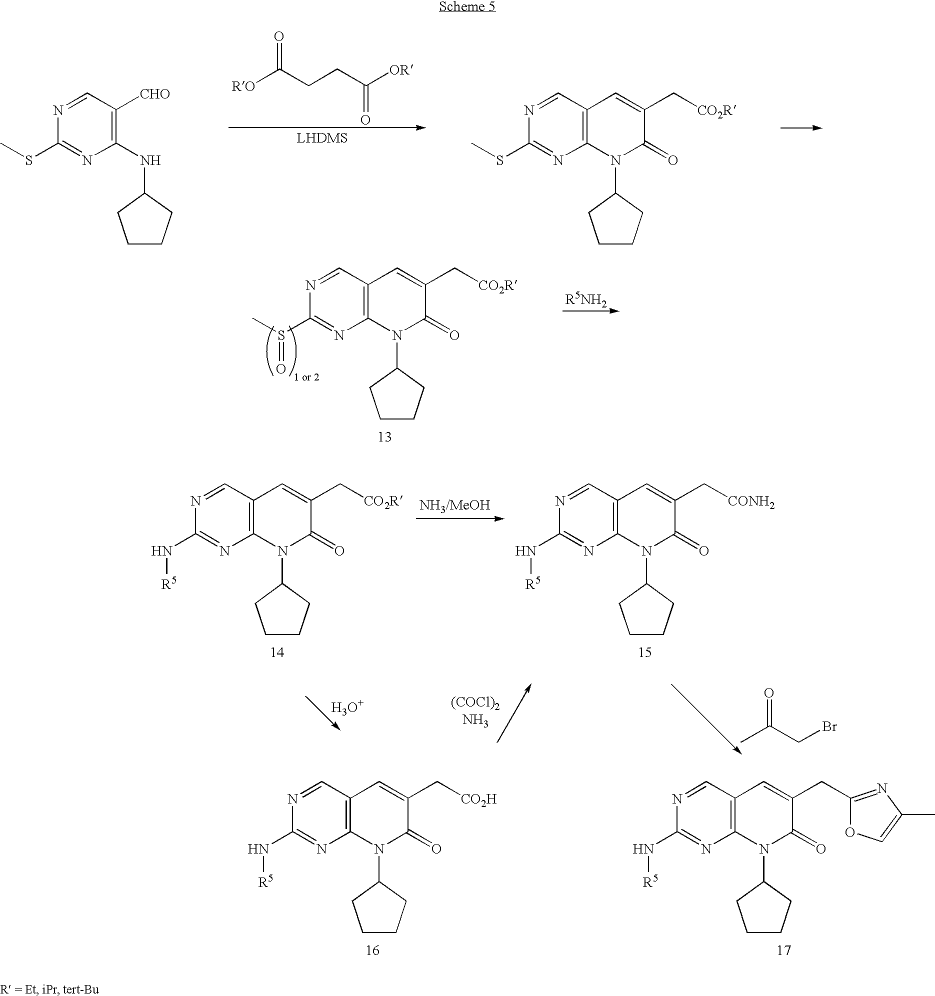

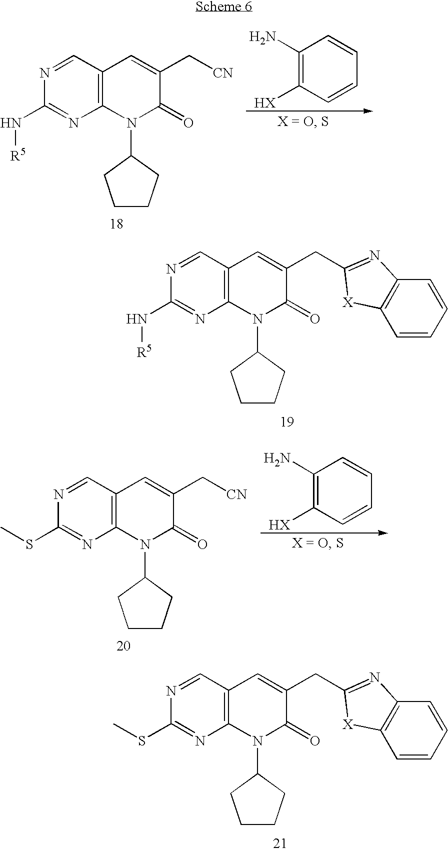

The compounds of the present invention can be formulated and administered in a wide variety of oral and parenteral dosage forms, including transdermal and rectal administration. It will be recognized to those skilled in the art that the following dosage forms may comprise as the active component, either a compound of Formula I or a corresponding pharmaceutically acceptable salt or solvate of a compound of Formula I.

This invention also comprises a pharmaceutical formulation comprising a therapeutically effective amount of a compound of Formula I together with a pharmaceutically acceptable carrier, diluent, or excipient therefor. For preparing pharmaceutical compositions with the compounds of the present invention, pharmaceutically acceptable carriers can be either a solid or liquid. Solid form preparations include powders, tablets, pills, capsules, cachets, suppositories, and dispensable granules. A solid carrier can be one or more substances which may also act as diluents, flavoring agents, binders, preservatives, tablet disintegrating agents, or an encapsulating material.

In powders, the carrier is a finely divided solid such as talc or starch which is in a mixture with the finely divided active component. In tablets, the active component is mixed with the carrier having the necessary binding properties in suitable proportions and compacted in the shape and size desired.

The formulations of this invention preferably contain from about 5% to about 70% or more of the active compound. Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. A preferred form for oral use are capsules, which include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient size molds, allowed to cool, and thereby to solidify.

Liquid form preparations include solutions, suspensions, and emulsions such as water or water/propylene glycol solutions. For parenteral injection, liquid preparations can be formulated in solution in aqueous polyethylene glycol solution, isotonic saline, 5% aqueous glucose, and the like. Aqueous solutions suitable for oral use can be prepared by dissolving the active component in water and adding suitable colorants, flavors, stabilizing and thickening agents as desired. Aqueous suspensions suitable for oral use can be made by dispersing the finely divided active component in water and mixing with a viscous material, such as natural or synthetic gums, resins, methylcellulose, sodium carboxymethylcellulose, or other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like. Waxes, polymers, microparticles, and the like can be utilized to prepare sustained-release dosage form s. Also, osmotic pumps can be employed to deliver the active compound uniformly over a prolonged period.

The pharmaceutical preparations of the invention are preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The therapeutically effective dose of a compound of Formula I will vary from approximately 0.01 mg/kg to approximately 100 mg/kg of body weight per day. Typical adult doses will be approximately 0.1 mg to approximately 3000 mg per day. The quantity of active component in a unit dose preparation may be varied or adjusted from approximately 0.1 mg to approximately 500 mg, preferably about 0.6 mg to 100 mg according to the particular application and the potency of the active component. The composition can, if desired, also contain other compatible therapeutic agents. A subject in need of treatment with a compound of Formula I is administered a dosage of about 0.6 to about 500 mg per day, either singly or in multiple doses over a 24-hour period. Such treatment may be repeated at successive intervals for as long as necessary.

The compounds of the present invention will typically be formulated with common excipients, diluents, and carriers to provide compositions that are well-suited for convenient administration to mammals. The following examples illustrate typical compositions that are provided in a further embodiment of this invention.

The compounds of the present invention may be freeze-dried, spray-dried, or evaporatively dried to provide a solid plug, powder, or film of crystalline or amorphous material. Microwave or radio frequency drying may be used for this purpose.

The compounds of the invention may be administered alone or in combination with other drugs and will generally be administered as a formulation in association with one or more pharmaceutically acceptable excipients. The term “excipient” is used herein to describe any ingredient other than the compound of the invention. The choice of excipient will to a large extent depend on the particular mode of administration.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

Tablet Formulation of the Compound of Example 40

Tablet Formulation

| Ingredient | Amount | |
|---|---|---|
| Compound of Example 40 | 50 | mg* |
| Lactose | 80 | mg |
| Cornstarch (for mix) | 10 | mg |
| Cornstarch (for paste) | 8 | mg |
| Magnesium Stearate (1%) | 2 | mg |
| | 150 | mg |

*Quantity adjusted in accordance with drug activity.

A compound of the present invention is mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for prevention and treatment of cancer.

Another composition of a typical tablet in accordance with the invention may comprise:

| Ingredient | % w/w |
|---|---|
| Compound of Example 40 | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of compounds of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

To a solution of 700 mL of propylene glycol and 200 mL of water for injection is added 20.0 g of the Compound of Example 8 of the present invention. The mixture is stirred and the pH is adjusted to 5.5 with hydrochloric acid. The volume is adjusted to 1000 mL with water for injection. The solution is sterilized, filled into 5.0 mL ampoules, each containing 2.0 mL (40 mg of compound), and sealed under nitrogen. The solution is administered by injection to a patient suffering from cancer and in need of treatment.

The compounds of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, 88 (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus compounds of the invention may be formulated in a more solid form for administration as an implanted depot providing long-term release of the active compound.

The compounds of the invention can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 10 mg of the compound of the invention per actuation and the actuation volume may vary from 1 μl to 100 μl. A typical formulation may comprise a compound of of this invention, propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the compound of the invention, a suitable powder base such as lactose or starch and a performance modifier such as Heucine, mannitol, or magnesium stearate.

In the case of dry powder inhalers and aerosols, the dosage unit is determined by means of a valve which delivers a metered amount. Units in accordance with the invention are typically arranged to administer a metered dose or “puff” appropriate for the disease state, age and size of the individual. The overall daily dose may be administered in a single dose or, more usually, as divided doses throughout the day.

Formulations for inhaled/intranasal administration may be formulated to be immediate and/or modified release. Modified release formulations include delayed-, sustained-, pulsed-, controlled dual-, targeted and programmed release.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

The compounds of the invention may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

The compounds of the invention may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds of the invention is typically in the range 0.1 mg to approximately 3000 mg depending, of course, on the mode of administration. For example, oral administration may require a total daily dose of from 10 mg to 3000 mg, while an intravenous dose may only require from 0.1 mg to 1000 mg/kg of body weight. The total daily dose may be administered in single or divided doses. These dosages are based on an average human subject having a weight of about 65 to 70 kg. The physician will readily be able to determine doses for subjects whose weight falls outside this range, such as infants and the elderly.

This invention provides a pharmaceutical composition for treating a disorder or condition selected from the group consisting of cell proliferative disorders, such as cancer, vascular smooth muscle proliferation associated with atherosclerosis, postsurgical vascular stenosis, restenosis, and endometriosis; infections, including viral infections such as DNA viruses like herpes and RNA viruses like HIV, and fungal infections; autoimmune diseases such as psoriasis, inflammation like rheumatoid arthritis, lupus, type 1 diabetes, diabetic nephropathy, multiple sclerosis, and glomerulonephritis, organ transplant rejection, including host versus graft disease.

The examples presented below are intended to illustrate particular embodiments of the invention, and are not intended to limit the scope of the specification or the claims in any way.

Those having skill in the art will recognize that the starting materials may be varied and additional steps employed to produce compounds encompassed by the present invention, as demonstrated by the following examples. The following examples are for illustrative purposes only and are not intended, nor should they be construed as limiting the invention in any manner. Those skilled in the art will appreciate that variations and modifications can be made without violating the spirit or scope of the invention.

### EXAMPLES

### Example 1

### (8-Cyclopentyl-2-methylsulfanyl-7-oxo-7,8-dihydro-pyrido[2,3-d]pyrimidin-6-yl)-acetic acid

To LiHMDS (21.03 mL, 126.4 mmol, 1.0 M, in THF) diluted in THF (75 mL) cooled to −78° C. was added diethyl succinate neat, this mixture was stirred for 10 min. To this mixture was added 4-cyclopentylamino-2-methylsulfanyl-pyrimidine-5-carbaldehyde (10 g, 42.14 mmol) dissolved in THF (50 mL). The reaction was stirred for an additional 30 min., then warmed to room temperature. After 16 hours the reaction mixture was diluted with ethyl acetate and water. The layers were separated and the aqueous layer was washed with ethyl acetate twice. A precipitate formed as the aqueous layer was acidified to pH 2 with conc. HCl. The precipitate was filtered off, then washed with hexanes to give (8-cyclopentyl-2-methylsulfanyl-7-oxo-7,8-dihydro-pyrido[2,3-d]pyrimidin-6-yl)-acetic acid as a white solid (8.48 g, 63%). ¹H NMR (400 MHz, CDCl₃) δ 8.63 (s, 1H), 7.62 (s,1H), 5.92-6.03 (m, 1H), 4.66 (s, 2H), 2.61 (s, 3H), 2.27-2.34 (m, 2H), 2.03-2.09 (m, 2H), 1.85-1.91 (m, 2H), 1.67-1.71 (m, 2H).

### Example 2

### (8-Cyclopentyl-2-methylsulfanyl-7-oxo-7,8-dihydro-pyrido[2,3-d]pyrimidin-6-yl)-acetic acid N′-acetyl-hydrazide

To a solution of (8-cyclopentyl-2-methylsulfanyl-7-oxo-7,8-dihydro-pyrido[2,3-d]pyrimidin-6-yl)-acetic acid (1.0 g, 3.13 mmol) in THF (15 mL) was added 1,1′-carbonyl diimidazole (609 mg, 3.76 mmol, 1.2 eq) and the solution was stirred at room temperature for 2 h. Acetic hydrazide (283 mg, 3.44 mmol, 1.1 eq) was added and after 1 h a precipitate formed. The mixture was stirred overnight at room temperature. The reaction mixture was filtered and the solid was washed with THF/Et₂O and dried in a vacuum oven to give (8-cyclopentyl-2-methylsulfanyl-7-oxo-7,8-dihydro-pyrido[2,3-d]pyrimidin-6-yl)-acetic acid N′-acetyl-hydrazide as a white solid (847 mg, 2.26 mmol, 72%). The structure was confirmed by NMR and mass spectrometry. MS: APCl: M+1: 376.0 (Exact Mass: 375.14). ¹H NMR (400 MHz, DMSO-d₆) δ 9.85 (s, 1H), 9.80 (s, 1H), 8.81 (s, 1H), 7.82 (s, 1H), 5.78-5.83 (m, 1H), 3.37 (s, 2H), 3.27 (s, 3H), 2.53 (s, 3H), 2.12-2.17 (m, 2H), 1.91-1.96 (m, 2H), 1.70-1.77 (m, 2H), 1.56-1.60 (m, 2H).

### Example 3

### Preparation of 8-Cyclopentyl-6-(5-methyl-[1,3,4]oxadiazol-2-ylmethyl)-2-methylsulfanyl-8H-pyrido[2,3-d]pyrimidin-7-one

A suspension of (8-cyclopentyl-2-methylsulfanyl-7-oxo-7,8-dihydro-pyrido[2,3-d]pyrimidin-6-yl)-acetic acid N′-acetyl-hydrazide (300 mg, 0.799 mmol) in POCl₃ (8 mL) was heated to 95° C. After 2 h, the reaction became homogeneous. The reaction mixture was heated for one more hour and then concentrated. The residue was partitioned between CH₂Cl₂ and H₂O. The aqueous layer was extracted with CH₂Cl₂. The combined organic layers were washed with H₂O and brine, dried over Na₂SO₄ and concentrated to give a yellow oil. Purification by chromatography (2% MeOH/ethyl acetate) gave 8-cyclopentyl-6-(5-methyl-[1,3,4]oxadiazol-2-ylmethyl)-2-methylsulfanyl-8H-pyrido[2,3-d]pyrimidin-7-one as a white solid (200 mg, 0.56 mmol, 70%). The structure was confirmed by NMR and mass spectrometry. MS: APCl: M+1: 358.0 (Exact Mass: 357.13).

### Example 4

### Preparation of 8-Cyclopentyl-2-methanesulfinyl-6-(5-methyl-[1,3,4]oxadiazol-2-ylmethyl)-8H-pyrido[2,3-d]pyrimidin-7-one

To a solution of 8-cyclopentyl-6-(5-methyl-[1,3,4]oxadiazol-2-ylmethyl)-2-methylsulfanyl-8H-pyrido[2,3-d]pyrimidin-7-one (195 mg, 0.546 mmol) in CH₂Cl₂ (4 mL) was added 2-benzenesulfonyl-3-phenyl-oxaziridine (214 mg, 0.818 mmol, 1.5 eq). The reaction was stirred overnight at room temperature. The reaction mixture was concentrated and purified by chromatography (5% MeOH/CH₂Cl₂) to give 8-cyclopentyl-2-methanesulfinyl-6-(5-methyl-[1,3,4]oxadiazol-2-ylmethyl)-8H-pyrido[2,3-d]pyrimidin-7-one as a white foam (190 mg, 0.509 mmol, 93%). %). ¹H NMR (400 MHz, CDCl₃) δ 8.59 (s, 1H), 7.62 (s, 1H), 5.95-5.99 (m, 1H), 4.15 (s, 2H), 2.63 (s, 3H), 2.54 (s, 3H), 2.36-2.29 (m, 2H), 2.03-2.19 (m, 2H), 1.86-1.91 (m, 2H), 1.68-1.72 (m, 2H).

### Example 5

### Preparation of 4-{4-[8-Cyclopentyl-6-(5-methyl-[1,3,4]oxadiazol-2-ylmethyl)-7-oxo-7,8-dihydro-pyrido[2,3-d]pyrimidin-2-ylamino]-phenyl}-piperazine-1-carboxylic acid tert-butyl ester

A mixture of 8-cyclopentyl-2-methanesulfinyl-6-(5-methyl-[1,3,4]oxadiazol-2-ylmethyl)-8H-pyrido[2,3-d]pyrimidin-7-one (185 mg, 0.495 mmol) and 4-(4-amino-phenyl)-piperazine-1-carboxylic acid tert-butyl ester (247 mg, 0.892 mmol, 1.8 eq) were dissolved in DMSO (2.5 mL) and heated at 80° C. for 2 days. Succinic anhydride (70 mg) was added to react with the excess aniline and the mixture was heated at 80° C. for 3 h. The reaction mixture was partitioned between ethyl acetate and H₂O. The organic layer was washed with saturated NaHCO₃ (2×), H₂O and brine, dried over Na₂SO₄ and concentrated to give an orange foam. Purification by liquid chromatography (2-4% MeOH/CH₂Cl₂) gave 4-{4-[8-cyclopentyl-6-(5-methyl-[1,3,4]oxadiazol-2-ylmethyl)-7-oxo-7,8-dihydro-pyrido[2,3-d]pyrimidin-2-ylamino]-phenyl}-piperazine-1-carboxylic acid tert-butyl ester as a yellow foam (202 mg, 0.344 mmol, 70%). The structure was confirmed by NMR and mass spectrometry. MS: APCl: M+1: 587.1 (Exact Mass: 586.30).

### Example 6

### Preparation of 8-Cyclopentyl-6-(5-methyl-[1,3,4]oxadiazol-2-ylmethyl)-2-(4-piperazin-1-yl-phenylamino)-8H-pyrido[2,3-d]pyrimidin-7-one

To a solution of 4-{4-[8-cyclopentyl-6-(5-methyl-[1,3,4]oxadiazol-2-ylmethyl)-7-oxo-7,8-dihydro-pyrido[2,3-d]pyrimidin-2-ylamino]-phenyl}-piperazine-1-carboxylic acid tert-butyl ester (198 mg, 0.337 mmol) in CH₂Cl₂ (0.7 mL) was added 2 N HCl in Et₂O (2.5 mL). The mixture was stirred overnight at room temperature and concentrated to give the title compound as a yellow solid (bis HCl salt, 199 mg, quantitative). The structure was confirmed by NMR and mass spectrometry. MS: APCl: M+1: 487.1 (Exact Mass: 486.25).

### Example 7

### Preparation of 6-Benzyl-8-cyclopentyl-2-methylsulfanyl-8H-pyrido[2,3-d]pyrimidin-7-one

To a solution of lithium bis(trimethylsilyl)amide (25.2 mL, 1 M in THF, 25.2 mmol, 3 eq) in THF (12 mL) cooled to −78° C. was added ethyl hydrocinnamate (4.45 mL, 25.2 mmol, 3 eq). After 20 minutes, 4-cyclopentylamino-2-methylsulfanyl-pyrimidine-5-carbaldehyde (2.0 g, 8.4 mmol) was added as a solution in THF (6 mL). The reaction mixture was stirred at −78° C. for 1 h and then allowed to warm to room temperature overnight. The reaction was quenched with saturated NH₄Cl and the mixture was extracted with ethyl acetate (2×). The organic layer was washed with H₂O, saturated NaHCO₃ and brine, dried over Na₂SO₄ and concentrated to give a green oil. 10% Ethyl acetate/hexanes was added and a white precipitate formed. The precipitate was collected by filtration, washed with 10% ethyl acetate/hexanes and dried to give 6-benzyl-8-cyclopentyl-2-methylsulfanyl-8H-pyrido[2,3-d]pyrimidin-7-one as a white solid (1.06 g, 3.02 mmol, 36%). The structure was confirmed by NMR and mass spectrometry. MS: APCl: M+1: 352.1 (Exact Mass: 351.14).

### Example 8

### Preparation of 6-Benzyl-8-cyclopentyl-2-methanesulfinyl-8H-pyrido[2,3-d]pyrimidin-7-one

6-Benzyl-8-cyclopentyl-2-methylsulfanyl-8H-pyrido[2,3-d]pyrimidin-7-one was oxidized according to Example 4. Chromatography (SiO₂, 65% EtOAc/CH₂Cl₂) gave 6-benzyl-8-cyclopentyl-2-methanesulfinyl-8H-pyrido[2,3-d]pyrimidin-7-one as a white foam (956 mg, 2.60 mmol, 87%). The structure was confirmed by NMR and mass spectrometry. MS: APCl: M+1: 368.0 (Exact Mass: 367.14).

### Example 9

### Preparation of 4-[4-(6-Benzyl-8-cyclopentyl-7-oxo-7,8-dihydro-pyrido[2,3-d]pyrimidin-2-ylamino)-phenyl]-piperazine-1-carboxylic acid tert-butyl ester

6-Benzyl-8-cyclopentyl-2-methanesulfinyl-8H-pyrido[2,3-d]pyrimidin-7-one (350 mg, 0.952 mmol) and 4-(4-amino-phenyl)-piperazine-1-carboxylic acid tert-butyl ester (474 mg, 1.71 mmol, 1.8 eq) in DMSO (4.5 mL) were reacted according to Example 5. The resulting brown foam was purified by chromatography (40% ethyl acetate/hexanes) to give 4-[4-(6-benzyl-8-cyclopentyl-7-oxo-7,8-dihydro-pyrido[2,3-d]pyrimidin-2-ylamino)-phenyl]-piperazine-1-carboxylic acid tert-butyl ester as a yellow foam (388 mg, 0.668 mmol, 70%). The structure was confirmed by NMR and mass spectrometry. MS: APCl: M+1: 581.2 (Exact Mass: 580.32).

### Example 10

### Preparation of 6-Benzyl-8-cyclopentyl-2-(4-piperazin-1-yl-phenylamino)-8H-pyrido[2,3-d]pyrimidin-7-one

4-[4-(6-Benzyl-8-cyclopentyl-7-oxo-7,8-dihydro-pyrido[2,3-d]pyrimidin-2-ylamino)-phenyl]-piperazine-1-carboxylic acid tert-butyl ester (375 mg, 0.660 mmol) was treated with 2 N HCl in Et₂O according to Example 6 to give the title compound as a yellow solid (HCl salt, 397 mg, quantitative). The structure was confirmed by NMR and mass spectrometry. MS: APCl: M+1: 481.1 (Exact Mass: 480.26).

### Example 11

### Preparation of 8-Cyclopentyl-2-methylsulfanyl-6-(1-phenyl-ethyl)-8H-pyrido[2,3-d]pyrimidin-7-one

The lithium enolate of methyl (R)-3-phenylbutyrate (Fluka, 4.7 mL, 26.9 mmol, 2 eq) was reacted with 4-cyclopentylamino-2-methylsulfanyl-pyrimidine-5-carbaldehyde according to Example 7. Purification by chromatography (10-15% ethyl acetate/hexanes) gave 8-cyclopentyl-2-methylsulfanyl-6-(1-phenyl-ethyl)-8H-pyrido[2,3-d]pyrimidin-7-one as a pink foam (3.90 g, 10.67 mmol, 79%). The structure was confirmed by NMR and mass spectrometry. MS: APCl: M+1: 366.1 (Exact Mass: 365.16).

### Example 12

### Preparation of 8-Cyclopentyl-2-methanesulfinyl-6-(1-phenyl-ethyl)-8H-pyrido[2,3-d]pyrimidin-7-one

8-Cyclopentyl-2-methylsulfanyl-6-(1-phenyl-ethyl)-8H-pyrido[2,3-d]pyrimidin-7-one (3.85 g, 10.53 mmol) was oxidized according to Example 4 to give, after chromatography (SiO₂, 70-75% ethyl acetate/CH₂Cl₂), 8-cyclopentyl-2-methanesulfinyl-6-(1-phenyl-ethyl-8H-pyrido[2,3-d]pyrimidin-7-one as a light orange foam (3.52 g, 9.23 mmol, 88%).

### Example 13

### Preparation of 4-{4-[8-Cyclopentyl-7-oxo-6-(1-phenyl-ethyl)-7,8-dihydro-pyrido[2,3-d]pyrimidin-2-ylamino]-phenyl}-piperazine-1-carboxylic acid tert-butyl ester

8-Cyclopentyl-2-methanesulfinyl-6-(1-phenyl-ethyl)-8H-pyrido[2,3-d]pyrimidin-7-one (1.645 g, 4.31 mmol) and 4-(4-aminophenyl)-piperazine-1-carboxylic acid tert-butyl ester (2.03 g, 7.32 mmol) in DMSO (20 mL) were reacted according to Example 5. The resulting brown foam was purified by liquid chromatography (40% ethyl acetate/hexanes) to give 4-{4-[8-cyclopentyl-7-oxo-6-(1-phenyl-ethyl)-7,8-dihydro-pyrido[2,3-d]pyrimidin-2-ylamino]-phenyl}-piperazine-1-carboxylic acid tert-butyl ester as a yellow foam (1.94 g, 3.28 mmol, 76%). The structure was confirmed by NMR and mass spectrometry. MS: APCl: M+1: 595.3 (Exact Mass: 594.33).

### Example 14

### Preparation of 8-Cyclopentyl-6-(1-phenyl-ethyl)-2-(4-piperazin-1-yl-phenylamino)-8H-pyrido[2,3-d]pyrimidin-7-one

4-{4-[8-Cyclopentyl-7-oxo-6-(1-phenyl-ethyl)-7,8-dihydro-pyrido[2,3-d]pyrimidin-2-ylamino]-phenyl}-piperazine-1-carboxylic acid tert-butyl ester (1.80 g, 3.03 mmol) was treated with 2 N HCl in Et₂O according to Example 6 to give 8-cyclopentyl-6-(1-phenyl-ethyl)-2-(4-piperazin-1-yl-phenylamino)-8H-pyrido[2,3-d]pyrimidin-7-one as a yellow solid (bis HCl salt, 1.63 g, 2.82 mmol, 93%). The structure was confirmed by NMR and mass spectrometry. MS: APCl: M+1: 495.2 (Exact Mass: 494.28).

### Example 15

### Preparation of 6-Benzoyl-8-cyclopentyl-2-methylsulfanyl-8H-pyrido[2,3-d]pyrimidin-7-one

To a solution of 4-cyclopentylamino-2-methylsulfanyl-pyrimidine-5-carbaldehyde (5.0 g, 21.1 mmol) in EtOH (60 mL) was added ethylbenzoylacetate (tech. 90%, 10.4 mmol, 60.0 mmol, approx. 3 eq) followed by piperidine (1 mL). The flask was fitted with a Dean-Stark trap and the reaction was heated to reflux overnight. The reaction mixture was cooled to 0° C. and a precipitate formed. The solid was collected by filtration, washed with EtOH and Et₂O and dried in a vacuum oven to give 6-benzoyl-8-cyclopentyl-2-methylsulfanyl-8H-pyrido[2,3-d]pyrimidin-7-one as a pale yellow fluffy solid (5.54 g, 15.2 mmol, 72%). The structure was confirmed by NMR and mass spectrometry. MS: APCl: M+1: 366.1 (Exact Mass: 365.12).

### Example 16

### Preparation of 6-Benzoyl-8-cyclopentyl-2-methanesulfinyl-8H-pyrido[2,3-d]pyrimidin-7-one

6-Benzoyl-8-cyclopentyl-2-methylsulfanyl-8H-pyrido[2,3-d]pyrimidin-7-one (3.0 g, 8.21 mmol) was oxidized according to Example 4 to give, after chromatography (SiO₂, 75-80% EtOAc/CH₂Cl₂), 6-benzoyl-8-cyclopentyl-2-methanesulfinyl-8H-pyrido[2,3-d]pyrimidin-7-one as a yellow foam (2.68 g, 7.03 mmol, 86%). The structure was confirmed by NMR and mass spectrometry. MS: APCl: M+1: 382.0 (Exact Mass: 381.11).

### Example 17

### Preparation of 4-[4-(6-Benzoyl-8-cyclopentyl-7-oxo-7,8-dihydro-pyrido[2,3-d]pyrimidin-2-ylamino)-phenyl]-piperazine-1-carboxylic acid tert-butyl ester

6-Benzoyl-8-cyclopentyl-2-methanesulfinyl-8H-pyrido[2,3-d]pyrimidin-7-one (1.936 g, 5.08 mmol) and 4-[4-aminophenyl]-piperazine-1-carboxylic acid tert-butyl ester (2.18 g, 7.86 mmol) in DMSO (20 mL) were reacted according to Example 5. The resulting brown foam was purified by chromatography (20-30% ethyl acetate/CH₂Cl₂) to give 4-[4-(6-benzoyl-8-cyclopentyl-7-oxo-7,8-dihydro-pyrido[2,3-d]pyrimidin-2-ylamino)-phenyl]-piperazine-1-carboxylic acid tert-butyl ester as a bright yellow-orange solid (2.47 g, 4.16 mmol, 82%). The structure was confirmed by NMR and mass spectrometry. MS: APCl: M+1: 595.2 (Exact Mass: 594.30).

### Example 18

### Preparation of 6-Benzoyl-8-cyclopentyl-2-(4-piperazin-1-yl-phenylamino)-8H-pyrido[2,3-d]pyrimidin-7-one

4-[4-(6-Benzoyl-8-cyclopentyl-7-oxo-7,8-dihydro-pyrido[2,3-d]pyrimidin-2-ylamino)-phenyl]-piperazine-1-carboxylic acid tert-butyl ester (200 mg, 0.336 mmol) was treated with 2 N HCl in Et₂O according to Example 6 to give 6-benzoyl-8-cyclopentyl-2-(4-piperazin-1-yl-phenylamino)-8H-pyrido[2,3-d]pyrimidin-7-one as an orange solid (bis HCl salt, 176 mg, 0.310 mmol, 92%). The structure was confirmed by NMR and mass spectrometry. MS: APCl: M+1: 495.1 (Exact Mass: 494.24).

### Example 19

### Preparation of 8-Cyclopentyl-2-methylsulfanyl-6-thiazol-2-ylmethyl-8H-pyrido[2,3-d]pyrimidin 7-one

The lithium enolate of 3-thiazol-2-yl-propionic acid ethyl ester (prepared from 2-thiazolecarboxaldehyde, 4.81 g, 26.0 mmol, 2 eq) was reacted with 4-cyclopentylamino-2-methylsulfanyl-pyrimidine-5-carbaldehyde (2.93 g, 12.38 mmol) according to Example 7. The residue after the work-up was dissolved in ethyl acetate and allowed to stand at room temperature. The crystals that formed were collected by filtration and washed with Et₂O to give 8-cyclopentyl-2-methylsulfanyl-6-thiazol-2-ylmethyl-8H-pyrido[2,3-d]pyrimidin-7-one as an off-white solid (1.33 g, 3.71 mmol, 30%). The structure was confirmed by NMR and mass spectrometry. MS: APCl: M+1: 359.2 (Exact Mass: 358.09).

### Example 20

### Preparation of 8-Cyclopentyl-2-methanesulfinyl-6-thiazol-2-ylmethyl-8H-pyrido[2,3-d]pyrimidin-7-one

8-Cyclopentyl-2-methylsulfanyl-6-thiazol-2-ylmethyl-8H-pyrido[2,3-d]pyrimidin-7-one (1.29 g, 3.60 mmol) was oxidized according to Example 4 to give, after chromatography (SiO₂, 5% MeOH/ethyl acetate and then 5% MeOH/CH₂Cl₂), 8-cyclopentyl-2-methanesulfinyl-6-thiazol-2-ylmethyl-8H-pyrido[2,3-d]pyrimidin-7-one as a yellow solid. The structure was confirmed by NMR and mass spectrometry. MS: APCl: M+1: 375.0 (Exact Mass: 374.09).

### Example 21

### Preparation of 4-[4-(8-Cyclopentyl-7-oxo-6-thiazol-2-ylmethyl-7,8-dihydro-pyrido[2,3-d]pyrimidin-2-ylamino)-phenyl]-piperazine-1-carboxylic acid tert-butyl ester

8-Cyclopentyl-2-methanesulfinyl-6-thiazol-2-ylmethyl-8H-pyrido[2,3-d]pyrimidin-7-one (300 mg, 0.801 mmol) and 4-[4-aminophenyl]-piperazine-1-carboxylic acid tert-butyl ester (400 mg, 1.44 mmol) in DMSO (4 mL) were reacted according to Example 5. The resulting brown foam was purified by two liquid chromatography steps, the first employing 100% ethyl acetate as the solvent and the then the product was repurified using 75% ethyl acetate/13% CH₃CN/12% hexanes as the solvent system to give 4-[4-(8-cyclopentyl-7-oxo-6-thiazol-2-ylmethyl-7,8-dihydro-pyrido[2,3-d]pyrimidin-2-ylamino)-phenyl]-piperazine-1-carboxylic acid tert-butyl ester as a yellow foam (303 mg, 0.516 mmol, 64%). The structure was confirmed by NMR and mass spectrometry. MS: APCl: M+1: 588.2 (Exact Mass: 587.27).

### Example 22

### Preparation of 8-Cyclopentyl-2-(4-piperazin-1-yl-phenylamino)-6-thiazol-2-ylmethyl-8H-pyrido[2,3-d]pyrimidin-7-one

4-[4-(8-Cyclopentyl-7-oxo-6-thiazol-2-ylmethyl-7,8-dihydro-pyrido[2,3-d]pyrimidin-2-ylamino)-phenyl]-piperazine-1-carboxylic acid tert-butyl ester (297 mg, 0.505 mmol) was treated with 2 N H Cl in Et₂O according to Example 6 to give 8-cyclopentyl-2-(4-piperazin-1-yl-phenylamino)-6-thiazol-2-ylmethyl-8H-pyrido[2,3-d]pyrimidin-7-one as a yellow solid (bis HCl salt, 288 mg, quantitative). The structure was confirmed by NMR and mass spectrometry. MS: APCl: M+1: 488.1 (Exact Mass: 487.22).

### Example 23

### Preparation of 3-(6-Benzyl-8-cyclopentyl-7-oxo-7,8-dihydro-pyrido[2,3-d]pyrimid-2-ylamino)-propionic acid

A suspension of 6-benzyl-8-cyclopentyl-2-methanesulfinyl-8H-pyrido[2,3-d]pyrimidin-7-one (0.150 g, 0.408 mmol), β-alanine (0.073 g, 0.816 mmol), and diisopropylethylamine (0.71 mL, 4.08 mmol) in acetonitrile (3 mL) was heated under reflux overnight. The suspension was partitioned between dichloromethane and dilute aqueous ammonium sulfate. The organic phase was separated, washed with brine, dried over magnesium sulfate and concentrated. The material was purified by chromotagraphy over silica gel with a gradient of 5-20% MeOH in dichloromethane over 15 minutes. The appropriate fractions were combined, concentrated and dried to give 3-(6-benzyl-8-cyclopentyl-7-oxo-7, 8-dihydro-pyrido[2,3-d]pyrimid-2-ylamino)-propionic acid as a white solid (0.0713 g, 44.6%). MS (M+1): Calc 393.2, Found 393.1

### Example 24

### Preparation of 6-Benzyl-8-cvclopentyl-2-[2-(1H-imidazol-4-yl)-ethylamino]-8H-pyrido[2,3-d]pyrimidin-7-one

A suspension of 6-benzyl-8-cyclopentyl-2-methanesulfinyl-8H-pyrido[2,3-d]pyrimidin-7-one (0.150 g, 0.408 mmol) and histamine (0.14 g, 1.26 mmol) in acetonitrile (2 mL) was heated under reflux for three hours. The suspension was concentrated and partitioned between dichloromethane and water. The organic phase was separated and dried over magnesium sulfate then filtered and concentrated. The material was purified by chromotagraphy over silica gel with a gradient of 0-20% MeOH in dichloromethane over 30 minutes. The appropriate fractions were combined, concentrated and dried to give 6-benzyl-8-cyclopentyl-2-[2-(1H-imidazol-4-yl)-ethylamino]-8H-pyrido[2,3-d]pyrimidin-7-one as a white solid. (0.1221 g, 72.2%). MS (M+1): Calc 415.2, Found 415.2

### Example 25

### Preparation of 8-cyclopentyl-2-methylsulfanyl-6-pyridin-2-ylmethyl-8H-pyrido[2,3-d]pyrimidin-7-one

To a stirring solution of 1,1,1,3,3,3-hexamethyldisilazane (2.8 mL, 13.47 mmol) in anhydrous THF (Baker, 15 mL) at −78° C. was added n-butyllithium (1.6 M in hexanes, 8.15 mL, 13.03 mmol) and this mixture was allowed to stir for 30 minutes at −78° C. To the reaction mixture was added 3-pyridin-2-yl-propionic acid ethyl ester (2.34 g, 13.03 mmol: J. Med. Chem, 1992, 36, (22), 3293-3299.) as a solution in THF (5 mL) via cannula transfer and the mixture was allowed to stir for 30 minutes at −78° C. The reaction was then charged with 4-cyclopentylamino-2-methylsulfanyl-pyrimidine-5-carbaldehyde (1.03 g, 4.34 mmol) as a solution in THF (5 mL) via cannula transfer and allowed to stir for 30 minutes at −78° C. The ice bath was removed and the reaction was allowed to warm to ambient temperature while stirring overnight. After 21 hours, the reaction mixture was diluted with saturated NH₄Cl and partitioned with ethyl acetate. The organic layer was washed twice with water and twice with saturated NaHCO₃. The organic layer was collected, dried over Na₂SO₄, filtered and concentrated under reduced pressure to afford a yellow oil. The afforded oil was purified via silica gel column chromatography in 9:1 to 6:1 to 2:1 dichloromethane/acetone to afford 8-cyclopentyl-2-methylsulfanyl-6-pyridin-2-ylmethyl-8H-pyrido[2,3-d]pyrimidin-7-one as a light yellow solid (0.67 g, 63.2%). (APCl+)=353.1.

### Example 26

### Preparation of 8-Cyclopentyl-2-(piperidin-4-ylamino)-6-pyridin-2-ylmethyl-8H-pyrido[2,3-d]pyrimidin-7-one

4-(8-Cyclopentyl-7-oxo-6-pyridin-2-ylmethyl-7,8-dihydro-pyrido[2,3-d]pyrimidin-2-ylamino)-piperidine-1-carboxylic acid tert-butyl ester was prepared from 8-cyclopentyl-2-methylsulfanyl-6-pyridin-2-ylmethyl-8H-pyrido[2,3-d]pyrimidin-7-one and 4-amino-piperidine-1-carboxylic acid tert-butyl ester according to Examples 4 and 5. To a stirring solution of 4-(8-cyclopentyl-7-oxo-6-pyridin-2-ylmethyl-7,8-dihydro-pyrido[2,3-d]pyrimidin-2-ylamino)-piperidine-1-carboxylic acid tert-butyl ester (0.234 g, 0.464 mmol) in dichloromethane (10 mL) at ambient temperature was added trifluoroacetic acid (2 mL) and the mixture was allowed to stir for 1 hour. The reaction mixture was concentrated under reduced pressure and the resulting residue was co-evaporated several times with dichloromethane, which afforded a yellow oil. Diethyl ether was added to the oil and the mixture was allowed to stand overnight, during which time solids had formed. The solids were filtered and washed several times with diethyl ether and placed in a vacuum oven at 40° C. to dry completely, which afforded 8-cyclopentyl-2-(piperidin-4-ylamino)-6-pyridin-2-ylmethyl-8H-pyrido[2,3-d]pyrimidin-7-one as a TFA salt (3 mole equivalents, 0.281 g, 81.2%). M.p.=138-140° C.

### Example 27

### Preparation of 8-Cyclopentyl-2-(1-methanesulfonyl-piperidin-4-ylamino)-6-pyridin-2-ylmethyl-8H-pyrido[2,3-d]pyrimidin-7-one

To a stirring suspension of 8-cyclopentyl-2-(piperidin-4-ylamino)-6-pyridin-2-ylmethyl-8H-pyrido[2,3-d]pyrimidin-7-one (0.112 g, 0.15 mmol, 3.0 eq. TFA salt) in dichloromethane at 0° C. was added triethylamine (0.10 mL, 0.708 mmol) and methanesulfonyl chloride (0.014 mL, 0.177 mmol) and the reaction mixture was allowed to warm to ambient temperature. After stirring for 2 hours, the reaction mixture was concentrated under reduced pressure and the afforded residue was partitioned between ethyl acetate and water. The organic layer was washed twice,with saturated NaHCO₃ and twice with brine. The organic layer was collected, dried over Na₂SO₄, filtered and concentrated under reduced pressure to afforded 8-cyclopentyl-2-(1-methanesulfonyl-piperidin-4-ylamino)-6-pyridin-2-ylmethyl-8H-pyrido[2,3-d]pyrimidin-7-one as a white solid (0.050 g, 58.8%). M.p.=220-223° C.

### Example 28

### Preparation of 8-Cyclopentyl-6-(hydroxy-phenyl-methyl)-5-methyl-2-methylsulfanyl-8H-pyrido[2,3-d]pyrimidin-7-one

8-Cyclopentyl-6-iodo-5-methyl-2-methylsulfanyl-8H-pyrido[2,3-d]pyrimidin-7-one (0.731 g, 1.82 mmol) was dissolved in anhydrous THF (18.6 mL) and cooled in a dry-ice acetone bath. Propylmagnesium chloride (1.82 mL, 2M solution in THF, 3.64 mmol) was added over 35 min to give an orange solution. HMPA (4 mL) was added followed by a cooled (−78° C.) solution of benzaldehyde (3.8 mmol) in THF containing 20% HMPA (8.4 mL total). The reaction mixture was kept cold for 2 h then allowed to warm to room temperature. The orange solution was diluted with water and extracted with ethyl acetate. The combined extracts were dried, filtered and concentrated in vacuo to give the crude product which was purified by silica gel chromatography (15% ethyl acetate in heptane). A second chromatographic step on basic alumina (15-50% ethyl acetate in heptane) provided 8-cyclopentyl-6-(hydroxy-phenyl-methyl)-5-methyl-2-methylsulfanyl-8H-pyrido[2,3-d]pyrimidin-7-one (0.684 g, 85%) as a milk-white viscous oil. ¹H NMR (300 MHz, CDCl₃) δ 8.86 (s, 1H), 7.42-7.30 (m, 5H), 6.03 (d, J=10.9 Hz, 1H), 5.95 (p, J=9.2 Hz, 1H), 5.68 (d, J=10.9 Hz, 1H), 2.55 (s, 3H), 2.45 (s, 3H), 2.08-1.95 (m, 2H), 1.90-1.77 (m, 2H), 1.70-1.60 (m, 2H).

### Example 29

### Preparation of 6-Benzyl-8-cyclopentyl-5-methyl-2-(4-piperazin-1-yl-phenylamino)-8H-pyrido[2,3-d]pyrimidin-7-one

4-{4-[8-Cyclopentyl-6-(hydroxy-phenyl-methyl)-5-methyl-7-oxo-7,8-dihydro-pyrido[2,3-d]pyrimidin-2-ylamino]-phenyl}-piperazine-1-carboxylic acid tert-butyl ester (0.065 g, 0.106 mmol) was treated with trifluoroacetic acid (0.55 mL) under nitrogen and the resulting dark red solution was cooled in an ice/water bath. Triethyl silane (34 μL, 0.212 mmol) was added dropwise and the heterogeneous mixture was stirred for 15 min. The bath then was removed and the reaction mixture was allowed to warm to room temperature, forming a purple solution. This solution was stirred at room temperature for 17 h, then evaporated to dryness. The residue was diluted with dichloromethane (5 mL) and reconcentrated in vacuo. This procedure was repeated twice more. The resulting residue then was triturated with ethyl ether to give 6-benzyl-8-cyclopentyl-5-methyl-2-(4-piperazin-1-yl-phenylamino)-8H-pyrido[2,3-d]pyrimidin-7-one (0.049 g, 94%) as a green-yellow solid. mp 114° C. ¹H NMR (300 MHz, DMSO-d₆) δ 9.75 (s, 1H), 8.83 (s, 1H), 8.74 (s, 2H), 7.58 (d, J=8.7 Hz, 2H), 7.25-7.15 (m, 5H), 6.98 (d, J=8.7 Hz, 2H), 5.95-5.90 (m, 1H), 3.96 (s, 2H), 3.28 (s, 8H), 2.38 (s, 3H), 2.23 (m, 2H), 1.91 (m, 2H), 1.77 (m, 2H), 1.59 (m, 2H).

### Example 30

### Preparation of 4-[4-(6-Benzoyl-8-cyclopentyl-5-methyl-7-oxo-7,8-dihydro-[pyrido[2,3-d]pyrimidin-2-ylamino)-phenyl]-piperazine-1-carboxylic acid tert-butyl ester

4-{4-[8-Cyclopentyl-6-(hydroxy-phenyl-methyl)-5-methyl-7-oxo-7,8-dihydro-pyrido[2,3-d]pyrimidin-2-ylamino]-phenyl}-piperazine-1-carboxylic acid tert-butyl ester (0.298 g, 0.488 mmol) and MnO₂ (0.42 g, 4.88 mmol) in anhydrous acetonitrile (10 mL) were heated to 80° C. for 17 h with stirring. The mixture was filtered warm and the flask and the residue were washed with hot methanol (2×5 mL). The combined organic filtrates were concentrated in vacuo to give an orange residue. This residue was chromatographed on silica gel (40% ethyl acetate in heptane) to 4-[4-(6-benzoyl-8-cyclopentyl-5-methyl-7-oxo-7,8-dihydro-pyrido[2,3-d]pyrimidin-2-ylamino)-phenyl]-piperazine-1-carboxylic acid tert-butyl ester (0.19 g, 37%) as a yellow solid. ¹H NMR (300 MHz, CDCl₃) δ 8.72 (s, 1H), 7.92-7.89 (d, J=8.4 Hz, 2H), 7.61-7.54 (m, 1H), 7.51-7.45 (m, 4H), 7.26 (s, 1H), 6.97 (d, J=8.9 Hz, 2H), 5.97-5.81 (p, J=8.7 Hz, 1H), 3.62-3.58 (m, 4H), 3.14-3.11 (m, 4H), 2.28 (s, 3H), 2.35-2.22 (m, 2H), 1.92-1.78 (m, 4H), 1.65-1.50 (m, 2H), 1.49 (s, 9H).

The following compounds were prepared from intermediates described above by following the procedures of Examples 4 to 7.

### Example 31

8-Cyclopentyl-6-(hydroxy-phenyl-methyl)-5-methyl-2-(4-piperazin-1-yl-phenylamino)-8H-pyrido[2,3-d]pyrimidin-7-one. Mp 185° C. (dec)

### Example 32

6-Benzoyl-8-cyclopentyl-5-methyl-2-(4-piperazin-1-yl-phenylamino)-8H-pyrido[2,3-d]pyrimidin-7-one. Mp 190° C. (dec)

### Example 33

6-Benzyl-8-cyclopentyl-2-[1-(4-methyl-piperazine-1-sulfonyl)-piperidin-4-ylamino]-8H-pyrido[2,3-d]pyrimidin-7-one. Mp 182° C.-183° C.

### Example 34

6-Benzyl-8-cyclopentyl-2-[1-(propane-2-sulfonyl)-piperidin-4-ylamino]-8H-pyrido[2,3-d]pyrimidin-7-one. Mp 210° C.-212° C.

### Example 35

8-Cyclopentyl-2-(1-methanesulfonyl-piperidin-4-ylamino)-6-pyridin-2-ylmethyl-8H-pyrido[2,3-d]pyrimidin-7-one. Mp 220° C.-223° C.

### Example 35

8-Cyclopentyl-2-[1-(propane-2-sulfonyl)-piperidin-4-ylamino]-6-pyridin-2-ylmethyl-8H-pyrido[2,3-d]pyrimidin-7-one. Mp 216° C.-219° C.

### Example 36

8-Cyclopentyl-2-(4-piperazin-1-yl-phenylamino)-6-pyridin-2-ylmethyl-8H-pyrido[2,3-d]pyrimidin-7-one. MS 482.1

### Example 37

8-Cyclopentyl-2-phenylamino-6-pyridin-2-ylmethyl-8H-pyrido[2,3-d]pyrimidin-7-one. MS 398.1

### Example 38

8-Cyclopentyl-6-(3,4-dimethoxy-benzyl)-2-(4-piperazin-1-yl-phenylamino)-8H-pyrido[2,3-d]pyrimidin-7-one. MS 541.2

### Example 39

8-Cyclopentyl-2-[4-(2-oxo-oxazolidin-3-yl)-phenylamino]-6-(1-phenyl-ethyl)-8H-pyrido[2,3-d]pyrimidin-7-one. MS 496.1

### Example 40

6-Benzyl-8-cyclopentyl-2-(4-dimethylamino-cyclohexylamino)-8H-pyrido[2,3-d]pyrimidin-7-one. Mp 167° C.-170° C.

### Example 41

6-Benzyl-2-cyclohexylamino-8-cyclopentyl-8H-pyrido[2,3-d]pyrimidin-7-one. Mp128° C.-130° C.

### Example 42

6-Benzyl-8-cyclopentyl-2-(2-hydroxy-ethylamino)-8H-pyrido[2,3-d]pyrimidin-7-one. Mp 177°C.-179° C.

### Example 43

6-Benzyl-2-benzylamino-8-cyclopentyl-8H-pyrido[2,3-d]pyrimidin-7-one. Mp 186° C.-188° C.

### Example 44

6-Benzyl-8-cyclopentyl-2-phenylamino-8H-pyrido[2,3-d]pyrimidin-7-one. Mp 161° C.-163° C.

### Example 45

6-Benzyl-8-cyclopentyl-2-(1-propyl-piperidin-4-ylamino)-8H-pyrido[2,3-d]pyrimidin-7-one. Mp 143° C.-145° C.

### Example 46

6-Benzyl-8-cyclopentyl-2-(piperidin-4-ylamino)-8H-pyrido[2,3-d]pyrimidin-7-one. Mp 75° C. (gums).

### Example 47

6-Benzyl-8-cyclopentyl-2-methylamino-8H-pyrido[2,3-d]pyrimidin-7-one. Mp 144° C.-146°C.

### Example 48

6-Benzyl-8-cyclopentyl-2-[(2-hydroxy-ethyl)-methyl-amino]-8H-pyrido[2,3-d]pyrimidin-7-one. Mp<60° C.

### Example 49

6-Benzyl-8-cyclopentyl-2-dimethylamino-8H-pyrido[2,3-d]pyrimidin-7-one. Mp 115° C.-117° C.

### Example 50

6-Benzyl-8-cyclopentyl-2-(3-imidazol-1-yl-propylamino)-8H-pyrido[2,3-d]pyrimidin-7-one. Mp 142° C.-144° C.

### Example 51

6-Benzyl-8-cyclopentyl-2-[2-(3H-imidazol-4-yl)-ethylamino]-8H-pyrido[2,3-d]pyrimidin-7-one. Mp 163° C.-165° C.

### Example 52

3-(6-Benzyl-8-cyclopentyl-7-oxo-7,8-dihydro-pyrido[2,3-d]pyrimidin-2-ylamino)-propionic acid. Mp 164° C.-170° C.

### Example 53

6-Benzyl-8-cyclopentyl-2-(5-piperazin-1-yl-pyridin-2-y!amino)-8H-pyrido[2,3-d]pyrimidin-7-one. MS 482.2

### Example 54

6-Benzyl-8-cyclopentyl-2-(1-methanesulfonyl-piperidin-4-ylamino)-8H-pyrido[2,3-d]pyrimidin-7-one. Mp 243° C.-245° C.

### Example 55

8-Cyclopentyl-6-(4-methoxy-benzyl)-2-(4-piperazin-1-yl-phenylamino)-8H-pyrido[2,3-d]pyrimidin-7-one. HPLC Rf (min) 15.63; MS 511.2; Purity 98%

### Example 56

8-Cyclopentyl-2-(4-piperazin-1-yl-phenylamino)-6-(4-trifluoromethyl-benzyl)-8H-pyrido[2,3-d]pyrimidin-7-one. HPLC Rf (min) 16.94; MS 549.2; Purity 98.8%

### Example 57

6-(4-Chloro-benzyl)-8-cyclopentyl-2-(4-piperazin-1 -yl-phenylamino)-8H-pyrido[2,3-d]pyrimidin-7-one. HPLC Rf (min) 16.68; Purity 93.5%

### Example 58

8-Cyclopentyl-6-(4-methyl-benzyl)-2-(4-piperazin-1-yl-phenylamino)-8H-pyrido[2,3-d]pyrimidin-7-one. HPLC Rf (min) 16.26; Purity 98.3%

### Example 59

8-Cyclopentyl-6-(4-methyl-benzyl)-2-(1-propyl-piperidin-4-ylamino)-8H-pyrido[2,3-d]pyrimidin-7-one. Mp 154° C.-155° C.

### Example 60

8-Cyclopentyl-6-(2-methyl-benzyl)-2-(1-propyl-piperidin-4-ylamino)-8H-pyrido[2,3-d]pyrimidin-7-one. Mp 149° C.-151° C.

### Example 61

8-Cyclopentyl-6-(3-methyl-benzyl)-2-(1-propyl-piperidin-4-ylamino)-8H-pyrido[2,3-d]pyrimidin-7-one. Mp 118° C.-120° C.

### Example 62

8-Cyclopentyl-6-(4-ethyl-benzyl)-2-(1-propyl-piperidin-4-ylamino)-8H-pyrido[2,3-d]pyrimidin-one. Mp 151° C.-153° C.

### Example 63

8-Cyclopentyl-6-(4-isopropyl-benzyl)-2-(1-propyl-piperidin-4-ylamino)-8H-pyrido[2,3-d]pyrimidin-7-one. Mp 156° C.-160° C.

### Example 64

8-Cyclopentyl-2-(4-dimethylamino-cyclohexylamino)-6-(2-methyl-benzyl)-8H-pyrido[2,3-d]pyrimidin-7-one. Mp 178° C.-183° C.

### Example 65

8-Cyclopentyl-2-(4-dimethylamino-cyclohexylamino)-6-(3-methyl-benzyl)-8H-pyrido[2,3-d]pyrimidin-7-one. Mp 191° C.-194° C.

### Example 66

8-Cyclopentyl-2-(4-dimethylamino-cyclohexylamino)-6-(4-methyl-benzyl)-8H-pyrido[2,3-d]pyrimidin-7-one. Mp 179° C.-184° C.

### Example 67

8-Cyclopentyl-2-(4-dimethylamino-cyclohexylamino)-6-(4-ethyl-benzyl)-8H-pyrido[2,3-d]pyrimidin-7-one. Mp 175° C.-181° C.

### Example 68

8-Cyclopentyl-2-(4-dimethylamino-cyclohexylamino)-6-(4-isopropyl-benzyl)-8H-pyrido[2,3-d]pyrimidin-7-one. Mp 174° C.-181° C.

### Example 69

8-Cyclopentyl-6-(2-methyl-benzyl)-2-(4-piperazin-1-yl-phenylamino)-8H-pyrido[2,3-d]pyrimidin-7-one. Mp 210° C. (Foams)

### Example 70

8-Cyclopentyl-6-(3-methyl-benzyl)-2-(4-piperazin-1-yl-phenylamino)-8H-pyrido[2,3-d]pyrimidin-7-one. Mp 200° C.-203° C.

### Example 71

8-Cyclopentyl-6-(4-ethyl-benzyl)-2-(4-piperazin-1-yl-phenylamino)-8H-pyrido[2,3-d]pyrimidin-7-one. Mp 175° C. (Foams).

### Example 72

8-Cyclopentyl-6-(4-isopropyl-benzyl)-2-(4-piperazin-1-yl-phenylamino)-8H-pyrido[2,3-d]pyrimidin-7-one. Mp 140° C.-145° C.

### Example 73

8-Cyclopentyl-6-(2-methyl-benzyl)-2-(2-morpholin-4-yl-ethylamino)-8H-pyrido[2,3-d]pyrimidin-7-one. Mp 134° C.-137° C.

### Example 74

8-Cyclopentyl-6-(2-methyl-benzyl)-2-(3-morpholin-4-yl-propylamino)-8H-pyrido[2,3-d]pyrimidin-7-one. Mp 118° C.-119° C.

### Example 75

8-Cyclopentyl-6-(4-methyl-benzyl)-2-(2-morpholin-4-yl-ethylamino)-8H-pyrido[2,3-d]pyrimidin-7-one. Mp 88° C.-91° C.

### Example 76

8-Cyclopentyl-6-(4-methyl-benzyl)-2-(3-morpholin-4-yl-propylamino)-8H-pyrido[2,3-d]pyrimidin-7-one. Mp 114° C.-116° C.

### Example 77

8-Cyclopentyl-6-(2-methyl-benzyl)-2-[3-(4-methyl-piperazin-1-yl)-propylamino]-8H-pyrido[2,3-d]pyrimidin-7-one. Mp >45° C.

### Example 78

8-Cyclopentyl-6-(4-methyl-benzyl)-2-[3-(4-methyl-piperazin-1-yl)-propylamino]-8H-pyrido[2,3-d]pyrimidin-7-one. Mp 123° C.-124° C.

### Example 79

6-Benzyl-8-cyclopentyl-2-(2-dimethylamino-ethylamino)-8H-pyrido[2,3-d]pyrimidin-7-one. HPLC Rf (min) 14.2; MS 392.2; Purity 99.8%

### Example 80

6-Benzyl-8-cyclopentyl-2-(3-dimethylamino-propylamino)-8H-pyrido[2,3-d]pyrimidin-7-one. HPLC Rf (min): 13.8; MS 406.2; Purity 96.6%

### Example 81

8-Cyclopentyl-2-(4-dimethylamino-butylamino)-6-(2-methyl-benzyl)-8H-pyrido[2,3-d]pyrimidin-7-one. HPLC Rf (min) 14.35; MS 434.3; Purity 94.9%

### Example 82

8-Cyclopentyl-2-(3-dimethylamino-propylamino)-6-(2-methyl-benzyl)-8H-pyrido[2,3-d]pyrimidin-7-one. MS 420.3.

### Example 83

8-Cyclopentyl-2-(3-dimethylamino-propylamino)-6-(4-methyl-benzyl)-8H-pyrido[2,3-d]pyrimidin-7-one. MS 420.3.

### Example 84

8-Cyclopentyl-2-(4-dimethylamino-cyclohexylamino)-6-(4-methyl-benzyl)-8H-pyrido[2,3-d]pyrimidin-7-one. HPLC Rf (min) 14.8; MS 460.3; Purity 97.9%

### Example 85

8-Cyclopentyl-2-(4-dimethylamino-cyclohexylamino)-6-(4-methyl-benzyl)-8H-pyrido[2,3-d]pyrimidin-7-one. HPLC Rf (min) 15.2; MS 460.3; Purity 97.6%

### Example 86

8-Cyclopentyl-2-(1-propyl-piperidin-4-ylamino)-6-thiazol-2-ylmethyl-8H-pyrido[2,3-d]pyrimidin-7-one. Mp 149° C.-152° C.

### Example 87

8-Cyclopentyl-2-(4-dimethylamino-cyclohexylamino)-6-thiazol-2-ylmethyl-8H-pyrido[2,3-d]pyrimidin-7-one. Mp 154° C.-157° C.

### Example 88

8-Cyclopentyl-2-(1-propyl-piperidin-4-ylamino)-6-pyridin-2-ylmethyl-8H-pyrido[2,3-d]pyrimidin-7-one. Mp 133° C.-134° C.

### Example 89

8-Cyclopentyl-2-(4-dimethylamino-butylamino)-6-pyridin-2-ylmethyl-8H-pyrido[2,3-d]pyrimidin-7-one. Mp 105° C.-107° C.

### Example 90

8-Cyclopentyl-2-(4-dimethylamino-butylamino)-6-thiazol-2-ylmethyl-8H-pyrido[2,3-d]pyrimidin-7-one. Mp 122° C.-124° C.

### Example 91

8-Cyclopentyl-2-[3-(4-methyl-piperazin-1-yl)-propylamino]-6-thiazol-2-ylmethyl-8H-pyrido[2,3-d]pyrimidin-7-one. HPLC Rf (min) 9.96; MS 468.2; Purity 99%

### Example 92

8-Cyclopentyl-2-[4-(4-methyl-piperazin-1-yl)-butylamino]-6-thiazol-2-ylmethyl-8H-pyrido[2,3-d]pyrimidin-7-one. HPLC Rf (min) 14.51; MS 482.2; Purity 99.8%

### Example 93

8-Cyclopentyl-6-(3-methyl-benzyl)-2-(piperidin-4-ylamino)-8H-pyrido[2,3-d]pyrimidin-7-one. HPLC Rf (min) 14.51; MS 418.2; Purity 93.2%

### Example 94

8-Cyclopentyl-6-(4-methyl-benzyl)-2-(piperidin-4-ylamino)-8H-pyrido[2,3-d]pyrimidin-7-one. HPLC Rf (min) 14.51; MS 418.2; Purity 94.1%

### Example 95

8-Cyclopentyl-2-[5-(4-methyl-piperazin-1-yl)-pentylamino]-6-thiazol-2-ylmethyl-8H-pyrido[2,3-d]pyrimidin-7-one. HPLC Rf (min) 10.78; MS 496.2; Purity 79.8%

### Example 96

8-Cyclopentyl-2-(5-morpholin-4-yl-pentylamino)-6-thiazol-2-ylmethyl-8H-pyrido[2,3-d]pyrimidin-7-one. HPLC Rf (min) 12.07; MS 483.2; Purity 96.4%

### Example 97

8-Cyclopentyl-2-(5-diisopropylamino-pentylamino)-6-thiazol-2-ylmethyl-8H-pyrido[2,3-d]pyrimidin-7-one. HPLC Rf (min) 13.22; MS 497.2; Purity 97.5%

### Example 98

8-Cyclopentyl-2-(3-diethylamino-propylamino)-6-thiazol-2-ylmethyl-8H-pyrido[2,3-d]pyrimidin-7-one. HPLC Rf (min) 11.74; MS 441.2; Purity 97.3%

### Example 99

8-Cyclopentyl-2-(6-dimethylamino-hexylamino)-6-thiazol-2-ylmethyl-8H-pyrido[2,3-d]pyrimidin-7-one. HPLC Rf (min) 12.4; MS 455.2; Purity 95.6%

### Example 100

8-Cyclopentyl-6-(5-methyl-[1,3,4]oxadiazol-2-ylmethyl)-2-[4-(4-methyl-piperazin-1-yl)-butylamino]-8H-pyrido[2,3-d]pyrimidin-7-one. MS 497.2.

### Example 101

8-Cyclopentyl-6-(5-methyl-[1,3,4]oxadiazol-2-ylmethyl)-2-[5-(4-methyl-piperazin-1-yl)-pentylamino]-8H-pyrido[2,3-d]pyrimidin-7-one. MS 495.2.

### Example 102

8-Cyclopentyl-6-(5-methyl-[1,3,4]oxadiazol-2-ylmethyl)-2-(piperidin-4-ylamino)-8H-pyrido[2,3-d]pyrimidin-7-one. HPLC Rf (min) 10.64; MS 410.2; Purity 88.4%

### Example 103

8-Cyclopentyl-6-(4-methyl-benzyl)-2-[4-(2-oxo-oxazolidin-3-yl)-phenylamino]-8H-pyrido[2,3-d]pyrimidin-7-one. Mp 289.2° C.-290° C.

EXAMPLES 104-154 were prepared by parallel synthesis according to the following Array Chemistry protocol:

Step A: Preparation of Reagent A

In a fume hood, prepare a 0.05 M solution of 6-benzyl-8-cyclopentyl-2-methanesulfinyl-8H-pyrido[2,3-d]pyrimidin-7-one or 8-cyclopentyl-2-methanesulfinyl-6-(1-phenyl-ethyl)-8H-pyrido[2,3-d]pyrimidin-7-one in DMSO.

Step B: Preparation of Reagent B

Prepare a 1.0 M solution of each amine to be used in this combinatorial array in DMSO. The amines used in this combinatorial array were:
Ethanolamine, 2-Methoxyethaylamine, 2-Morpholin-4-yl-ethylamine, 3-Methylbutylamine, 2-(2-Amino-ethoxy)-ethanol, N1, N1-diethyl ethane diamine, 2-aminopropan-1-ol, isopropylamine, 1,3-propane diamine, cyclopentylamine, 3-aminopropane-1,2-diol, 1,3-dimethyl butylamine, N1, N1-dimethylpropane-1,2-diamine, 1-ethynylcyclohexylamine, 3-Pyrrolidin-1-yl-propylamine, 3-Morpholin-4-yl-propylamine, 2-Methoxy-1-methyl-ethylamine, 3-Methoxy-propylamine, (4-Amino-cyclohexyl)-acetic acid, 4-Amino-cyclohexanol, (1-Ethyl-piperidin-3-yl)-methyl-amine, 3,3-Dimethyl-butylamine, 3-Methylsulfanyl-propylamine, 2-Methoxy-1-methyl-ethylamine, cyclohexylamine, 4-amino-cyclohexylamine, 4-methylcyclohexylamine, 4-Amino-cyclohexanecarboxylic acid, N,N-Diethyl-cyclohexane-1,4-diamine, (4-Amino-cyclohexyl)-acetic acid, 4-aminocyclohexanol, 1-Ethyl-piperidin-3-ylamine, 1-Propyl-piperidin-4-ylamine, 3-Piperidin-1-yl-propylamine, N,N-Dimethyl-cyclohexane-1,4-diamine.
   However, it would be clear to one of skill in the art that other amines could be substituted for the those listed above.
   Step C:

Add 2.0 mL vials (0.1 mmol) each of Reagent A to separate 8 mL vial. Add a different Reagent B (0.200 mL, 0.2 mmol) to each vial. Shake the vials for 4 h at 100° C. Analyze reaction progress in representative samples by LCMS.

Step D:

Cool the vials to 25° C. then concentrate the crude samples in a Genevac centrifugal evaporator.

Step E:

Purify all samples by RP-HPLC. Using a 50×4.6 mm MetaChem Polaris C18 column, 3 μm. The Mobile phase was 80% Solvent A (Water+0.1% Formic acid): 20% Solvent B (Acetonitrile+0.1% Formic acid) to 2% solvent A: 98% B over 5 min.

### Example 104

6-Benzyl-8-cyclopentyl-2-(2-hydroxy-ethylamino)-8H-pyrido[2,3-d]pyrimidin-7-one. MS 365; LC/MS Rf-1.24; Purity 99.18%

### Example 105

6-Benzyl-8-cyclopentyl-2-(2-methoxy-ethylamino)-8H-pyrido[2,3-d]pyrimidin- 7-one. MS 379; LC/MS Rf-3.4; Purity 100%

### Example 106

6-Benzyl-8-cyclopentyl-2-(2-morpholin-4-yl-ethylamino)-8H-pyrido[2,3-d]pyrimidin-7-one. MS 379; LC/MS Rf-1.51; Purity 100%

### Example 107

6-Benzyl-8-cyclopentyl-2-(3-methyl-butylamino)-8H-pyrido[2,3-d]pyrimidin-7-one. MS 391; LC/MS Rf-4.2; Purity 100%

### Example 108

6-Benzyl-8-cyclopentyl-2-[2-(2-hydroxy-ethoxy)-ethylamino]-8H-pyrido[2,3-d]pyrimidin-7-one. MS 409; LC/MS Rf-2.89; 100% purity.
Example 109
6-Benzyl-8-cyclopentyl-2-(2-diethylamino-ethylamino)-8H-pyrido[2,3-d]pyrimidin-7-one. MS 420; LC/MS Rf-2.24; Purity 100%

### Example 110

6-Benzyl-8-cyclopentyl-2-(2-hydroxy-1-methyl-ethylamino)-8H-pyrido[2,3-d]pyrimidin-7-one. MS 379; LC/MS Rf-3.03; Purity 100%

### Example 111

6-Benzyl-8-cyclopentyl-2-isopropylamino-8H-pyrido[2,3-d]pyrimidin-7-one. MS 363; LC/MS Rf-1.68; Purity 100%

### Example 112

2-(2-Amino-ethylamino)-6-benzyl-8-cyclopentyl-8H-pyrido[2,3-d]pyrimidin-7-one. MS 364; LC/MS Rf-0.86; Purity 87.44%

### Example 113

6-Benzyl-8-cyclopentyl-2-cyclopentylamino-8H-pyrido[2,3-d]pyrimidin-7-one. MS 389; LC/MS Rf-1.79; Purity 100%

### Example 114

6-Benzyl-8-cyclopentyl-2-(2,3-dihydroxy-propylamino)-8H-pyrido[2,3-d]pyrimidin-7-one. MS 395; LC/MS Rf-1.12; Purity 99.33%

### Example 115

6-Benzyl-8-cyclopentyl-2-(1,3-dimethyl-butylamino)-8H-pyrido[2,3-d]pyrimidin-7-one. MS 405; LC/MS Rf-1.9; Purity 100%

### Example 116

6-Benzyl-8-cyclopentyl-2-(2-dimethylamino-1-methyl-ethylamino)-8H-pyrido[2,3-d]pyrimidin-7-one. MS 406; LC/MS Rf-0.89; Purity 99.28%

### Example 117

6-Benzyl-8-cyclopentyl-2-(1-ethynyl-cyclohexylamino)-8H-pyrido[2,3-d]pyrimidin-7-one. MS 427; LC/MS Rf-1.83; Purity 98.56%

### Example 118

6-Benzyl-8-cyclopentyl-2-(3-pyrrolidin-1-yl-propylamino)-8H-pyrido[2,3-d]pyrimidin-7-one. MS 432; LC/MS Rf-0.92; Purity 91.02%

### Example 119

6-Benzyl-8-cyclopentyl-2-(3-morpholin-4-yl-propylamino)-8H-pyrido[2,3-d]pyrimidin-7-one. MS 448; LC/MS Rf-0.87; Purity 97.67%

### Example 120

6-Benzyl-8-cyclopentyl-2-(2-methoxy-1-methyl-ethylamino)-8H-pyrido[2,3-d]pyrimidin-7-one. MS 393; LC/MS Rf-1.64; Purity 98.28%

### Example 121

6-Benzyl-8-cyclopentyl-2-(3-methoxy-propylamino)-8H-pyrido[2,3-d]pyrimidin-7-one. MS 393; LC/MS Rf-1.55; Purity 100%

### Example 122

[4-(6-Benzyl-8-cyclopentyl-7-oxo-7,8-dihydro-pyrido[2,3-d]pyrimidin-2-ylamino)-cyclohexyl]-acetic acid. MS 461; LC/MS Rf-1.46; Purity 94.04%

### Example 123

6-Benzyl-8-cyclopentyl-2-(4-hydroxy-cyclohexylamino)-8H-pyrido[2,3-d]pyrimidin-7-one. MS 419; LC/MS Rf-1.34; Purity 96.02%

### Example 124

6-Benzyl-8-cyclopentyl-2-(1-ethyl-piperidin-3-ylamino)-8H-pyrido[2,3-d]pyrimidin-7-one. MS 432; LC/MS Rf-0.89; Purity 98.27%

### Example 125

8-Cyclopentyl-2-(2-methoxy-ethylamino)-6-(1-phenyl-ethyl)-8H-pyrido[2,3-d]pyrimidin-7-one. MS 393; LC/MS Rf-1.56; Purity 99.31%

### Example 126

8-Cyclopentyl-2-(3-methyl-butylamino)-6-(1-phenyl-ethyl)-8H-pyrido[2,3-d]pyrimidin-7-one. MS 405; LC/MS Rf-1.88; Purity 100%

### Example 127

8-Cyclopentyl-2-[2-(2-hydroxy-ethoxy)-ethylamino]-6-(1-phenyl-ethyl)-8H-pyrido[2,3-d]pyrimidin-7-one. MS 423; LC/MS Rf-1.33; Purity 99.39%

### Example 128

8-Cyclopentyl-2-(2-diethylamino-ethylamino)-6-(1-phenyl-ethyl)-8H-pyrido[2,3-d]pyrimidin-7-one. MS 434; LC/MS Rf-0.92; Purity 97.55%

### Example 129

8-Cyclopentyl-2-isopropylamino-6-(1-phenyl-ethyl)-8H-pyrido[2,3-d]pyrimidin-7-one. MS 377; LC/MS Rf-1.72; Purity 95.73%

### Example 130

8-Cyclopentyl-2-(2-hydroxy-1-methyl-ethylamino)-6-(1-phenyl-ethyl)-8H-pyrido[2,3-d]pyrimidin-7-one. MS 393; LC/MS Rf-1.39; Purity 100%

### Example 131

8-Cyclopentyl-2-(2-morpholin-4-yl-ethylamino)-6-(1-phenyl-ethyl)-8H-pyrido[2,3-d]pyrimidin-7-one. MS 448; LC/MS Rf-0.9; Purity 100%

### Example 132

2-(2-Amino-ethylamino)-8-cyclopentyl-6-(1-phenyl-ethyl)-8H-pyrido[2,3-d]pyrimidin-7-one. MS 378; LC/MS Rf-0.89; Purity 92.3%

### Example 133

8-Cyclopentyl-2-cyclopentylamino-6-(1-phenyl-ethyl)-8H-pyrido[2,3-d]pyrimidin-7-one. MS 403; LC/MS Rf-1.83; Purity 100%

### Example 134

8-Cyclopentyl-2-(1,3-dimethyl-butylamino)-6-(1-phenyl-ethyl)-8H-pyrido[2,3-d]pyrimidin-7-one. MS 419; LC/MS Rf-1.93; Purity 100%

### Example 135

8-Cyclopentyl-2-(3,3-dimethyl-butylamino)-6-(1-phenyl-ethyl)-8H-pyrido[2,3-d]pyrimidin-7-one. MS 419; LC/MS Rf-1.93; Purity 100%

### Example 136

8-Cyclopentyl-2-(2-dimethylamino-1-methyl-ethylamino)-6-(1-phenyl-ethyl)-8H-pyrido[2,3-d]pyrimidin-7-one. MS 420; LC/MS Rf-0.91; Purity 98.82%

### Example 137

8-Cyclopentyl-2-(3-methylsulfanyl-propylamino)-6-(1-phenyl-ethyl)-8H-pyrido[2,3-d]pyrimidin-7-one. MS 423; LC/MS Rf-1.7; Purity 97.1%

### Example 138

8-Cyclopentyl-2-(1,4-dimethyl-pentylamino)-6-(1-phenyl-ethyl)-8H-pyrido[2,3-d]pyrimidin-7-one. MS 433; LC/MS Rf-2; Purity 100%

### Example 139

8-Cyclopentyl-2-(1-ethynyl-cyclohexylamino)-6-(1-phenyl-ethyl)-8H-pyrido[2,3-d]pyrimidin-7-one. MS 441; LC/MS Rf-1.87; Purity 98.38%

### Example 140

8-Cyclopentyl-2-(3-morpholin-4-yl-propylamino)-6-(1-phenyl-ethyl)-8H-pyrido[2,3-d]pyrimidin-7-one. MS 462; LC/MS Rf-0.9; Purity 97.75%

### Example 141

8-Cyclopentyl-2-(2-methoxy-1-methyl-ethylamino)-6-(1-phenyl-ethyl)-8H-pyrido[2,3-d]pyrimidin-7-one. MS 407; LC/MS Rf-1.65; Purity 96.78%

### Example 142

8-Cyclopentyl-2-(2-hydroxy-ethylamino)-6-(1-phenyl-ethyl)-8H-pyrido[2,3-d]pyrimidin-7-one. MS 379; LC/MS Rf-1.31; Purity 99.32%

### Example 143

2-Cyclohexylamino-8-cyclopentyl-6-(1-phenyl-ethyl)-8H-pyrido[2,3-d]pyrimidin-7-one. MS 417; LC/MS Rf-1.9; Purity 100%

### Example 144

2-(4-Amino-cyclohexylamino)-8-cyclopentyl-6-(1-phenyl-ethyl)-8H-pyrido[2,3-d]pyrimidin-7-one. MS 432; LC/MS Rf-0.93; Purity 97.53%

### Example 145

8-Cyclopentyl-2-(3-methoxy-propylamino)-6-(1-phenyl-ethyl)-8H-pyrido[2,3-d]pyrimidin-7-one. MS 407; LC/MS Rf-1.6; Purity 100%

### Example 146

8-Cyclopentyl-2-(4-methyl-cyclohexylamino)-6-(1-phenyl-ethyl)-8H-pyrido[2,3-d]pyrimidin-7-one. MS 431; LC/MS Rf-1.97; Purity 95.62%

### Example 147

4-[8-Cyclopentyl-7-oxo-6-(1-phenyl-ethyl)-7,8-dihydro-pyrido[2,3-d]pyrimidin-2-ylamino]-cyclohexanecarboxylic acid. MS 461; LC/MS Rf-1.43; Purity 93.98%

### Example 148

8-Cyclopentyl-2-(4-diethylamino-cyclohexylamino)-6-(1-phenyl-ethyl)-8H-pyrido[2,3-d]pyrimidin-7-one. MS 488; LC/MS Rf-1.00; Purity 98.08%

### Example 149

{4-[8-Cyclopentyl-7-oxo-6-(1-phenyl-ethyl)-7,8-dihydro-pyrido[2,3-d]pyrimidin-2-ylamino]-cyclohexyl}-acetic acid. MS 475; LC/MS Rf-1.51; Purity 94.74%

### Example 150

8-Cyclopentyl-2-(4-hydroxy-cyclohexylamino)-6-(1-phenyl-ethyl)-8H-pyrido[2,3-d]pyrimidin-7-one. MS 433; LC/MS Rf-1.39; Purity 97.81 %

### Example 151

8-Cyclopentyl-2-(1-ethyl-piperidin-3-ylamino)-6-(1-phenyl-ethyl)-8H-pyrido[2,3-d]pyrimidin-7-one. MS 446; LC/MS Rf-0.91; Purity 94.32%

### Example 152

8-Cyclopentyl-6-(1-phenyl-ethyl)-2-(1-propyl-piperidin-4-ylamino)-8H-pyrido[2,3-d]pyrimidin-7-one. MS 460; LC/MS Rf-0.96; Purity 100%

### Example 153

8-Cyclopentyl-6-(1-phenyl-ethyl)-2-(3-piperidin-1-yl-propylamino)-8H-pyrido[2,3-d]pyrimidin-7-one. MS 460; LC/MS Rf-0.94; Purity 98.56%

### Example 154

8-Cyclopentyl-2-(4-dimethylamino-cyclohexylamino)-6-(1-phenyl-ethyl)-8H-pyrido[2,3-d]pyrimidin-7-one. MS 460; LC/MS Rf-0.96; Purity 99.49%

The compounds of Examples 61-64, 85-87, 90, 91, 97-105 and 108 were purified by HPLC on a Vydac C18 4.6×25 μm column. The mobile phase was 90% solvent A (0.1% TFA in Water): 10% solvent B (0.1% TFA in MeCN) to 5% solvent A: 95% solvent B over 22 minutes at a flow rate of 1 mL/min.

### Example 155

4-[4-(8-Cyclopentyl-7-oxo-6-pyridin-2-ylmethyl-7,8-dihydro-pyrido[2,3-d]pyrimidin-2-ylamino)-phenyl]-piperazine-1-carboxylic acid tert-butyl ester. LC/MS Rf 582.2

### Example 156

Biological Assays

To determine the inhibitory potency and selectivity of compounds of the present invention against Cdk4 and related kinases, compounds were evaluated in standard assays routinely used to measure inhibition of cyclin-dependent kinase enzymes and other protein kinases (see for example D. W. Fry et al., J. Biol. Chem. 2001, 276, 16617-16623). The assays were carried out as described below.

Assay for Inhibition of Cdk2/Cyclin A

Cdk2 enzyme assays for IC₅₀ determinations and kinetic evaluation are performed as follows. 96-well filter plates (Millipore MADVN6550) are used. The final assay volume is 0.1 mL containing buffer A (20 mM TRIS (tris[hydroxymethyl]aminomethane) (pH 7.4), 50 mM NaCl, 1 mM dithiothreitol, 10 mM MgCl₂), 12 mM ATP containing 0.25 μCi [³²P]ATP, 20 ng Cdk2/cyclin A, 1 μg retinoblastoma protein, and the test compound at appropriate dilutions in buffer A (Buffer A alone without added test compound was employed as a control for no inhibition. Buffer A containing excess EDTA was used to determine the level of background ³²P in the absence of enzyme activity). All components except the ATP are added to the wells, and the plate is placed on a plate mixer for 2 minutes. The reaction is initiated by addition of [³²P]ATP, and the plate is incubated at 25° C. for 15 minutes. The reaction is terminated by addition of 0.1 mL 20% TCA. The plate is kept at 4° C. for at least 1 hour to allow the substrate to precipitate. The wells are then washed five times with 0.2 mL 10% TCA, and ³²P incorporation is determined with a beta plate counter (Wallac Inc., Gaithersburg, Md.). The IC₅₀ of the test compound was determined using the median effect method (Chou, T-C and Talalay, P. Applications of the median effect principle for the assessment of low-dose risk of carcinogens and for the quantitation of synergism and antagonism of chemotherapeutic agents. In: New Avenues in Developmental Cancer Chemotherapy (Eds. Harrap, K. T. and Connors, T. A.), pp. 37-64. Academic Press, New York, 1987).

Assay for Inhibition of Cdk4/Cyclin D

The Cdk4 enzyme assay for IC₅₀ determination and kinetic evaluation is performed as follows. 96-well filter plates (Millipore MADVN6550) are used. The total volume is 0.1 mL containing buffer A (20 mM TRIS (tris[hydroxymethyl]aminomethane) (pH 7.4), 50 mM NaCl, 1 mM dithiothreitol, 10 mM MgCl₂), 25 μM ATP containing 0.25 μCi [³²P]ATP, 20 ng Cdk4, 1 μg retinoblastoma protein and the test compound at appropriate dilutions in buffer A. Buffer A alone without added test compound was employed as a control for no inhibition. Buffer A containing excess EDTA was used to determine the level of background ³²P in the absence of enzyme activity. All components except the ATP are added to the wells, and the plate is placed on a plate mixer for 2 minutes. The reaction is started by adding [³²P]ATP, and the plate is incubated at 25° C. for 15 minutes. The reaction is terminated by addition of 0.1 mL 20% trichloroacetic acid (TCA). The plate is kept at 4° C. for at least 1 hour to allow the substrate to precipitate. The wells are then washed five times with 0.2 mL 10% TCA, and ³²P incorporation is determined with a beta plate counter (Wallac Inc., Gaithersburg, Md.). The IC₅₀ of the test compound was determined using the median effect method (Chou, T-C and Talalay, P. Applications of the median effect principle for the assessment of low-dose risk of carcinogens and for the quantitation of synergism and antagonism of chemotherapeutic agents. In: New Avenues in Developmental Cancer Chemotherapy (Eds. Harrap, K. T. and Connors, T. A.), pp. 37-64. Academic Press, New York, 1987).

Assay for Inhibition of FGFr

For FGF receptor (FGFr) tyrosine kinase assays 96-well plates (100 μL/incubation/well), and conditions are optimized to measure the incorporation of ³²P from [γ³²P]ATP into a glutamate-tyrosine co-polymer substrate. Briefly, to each well is added 82.5 μL incubation buffer B (25 mM Hepes (pH 7.0), 150 mM NaCl, 0.1% Triton X-100, 0.2 mM PMSF, 0.2 mM Na₃VO₄, 10 mM MnCl₂) and 750 μg/mL Poly (4:1) glutamate-tyrosine followed by 2.5 μL of the test compound in buffer B and 5 μL of a 7.5 μg/μL FGFr solution to initiate the reaction. Following a 10-minute incubation at 25° C., 10 mL [γ³²P]ATP (0.4 μCi plus 50 μM ATP) is added to each well, and samples are incubated for an additional 10 minutes at 25° C. The reaction is terminated by the addition of 100 μL 30% trichloroacetic acid (TCA) containing 20 mM sodium pyrophosphate and precipitation of material onto glass fiber mats (Wallac). Filters are washed three times with 15% TCA containing 100 mM sodium pyrophosphate, and the radioactivity retained on the filters is counted in a Wallac 1250 Betaplate reader. Nonspecific activity is defined as radioactivity retained on the filters following incubation of samples with buffer alone (no enzyme). Specific enzymatic activity (enzyme plus buffer) is defined as total activity minus nonspecific activity. The concentration of a test compound that inhibited specific activity by 50% (IC₅₀) is determined based on the inhibition curve.

Assay for Inhibition of PDGFr

Enzyme assays for IC₅₀ determinations were performed in 96-well filter plates (Millipore MADVNˆ%%), Millipore, Bedford, Mass.). The total volume was 100 μL/incubation/well) containing (20 mM Hepes (pH 7.4), 50 μM sodium vanadate, 40 mM magnesium chloride, 10 mM Manganese chloride, 10 μM adenosine triphosphate (ATP) containing [γ³²P]ATP (0.5 μCi, 20 μg of polyglutamic acid/tyrosine (Sigma Chemical Co., St. Louis, Mo.), 10 ng of the intracellular domain of PDGF receptor and appropriate dilutions of the inhibitors. All components except the ATP were added to the well and the plate incubated with shaking for 10 min at 25° C. The reaction is started by adding [γ³²P]ATP, and the plate is incubated for 10 min at 25° C. The reaction is terminated by the addition of 100 μL of 20% trichloroacetic acid (TCA). The plate is kept at 4° C. for at least 15 minutes to allow the substrate to precipitate. The wells were washed 5 times with 0.2 ml of 10% TCA and the radioactivity retained on the filters is counted in a Wallac 1250 Betaplate reader. Nonspecific activity is defined as radioactivity retained on the filters following incubation of samples with buffer alone (no enzyme). Specific enzymatic activity (enzyme plus buffer) is defined as total activity minus nonspecific activity. The concentration of a test compound that inhibited specific activity by 50% (IC₅₀) is determined based on the inhibition curve.

Results from the foregoing assays for several compounds of the present invention are presented in Table 1.

TABLE 1

| Compound of | CDK2/A IC₅₀ | CDK4/D | FGFr | PDGFr IC₅₀ |
|---|---|---|---|---|
| EXAMPLE No. | (□M) | IC₅₀ (□M) | IC₅₀ (□M) | (□M) |
| 31 | >5 | 0.019 | 0.095 | 0.538 |
| 29 | >5 | 0.007 | 3.095 | 1.035 |
| 32 | >5 | 0.012 | 2.430 | 2.710 |
| 33 | 4.9 | 0.002 | >5 | >5 |
| 34 | 3.5 | 0.001 | >5 | >5 |
| 26 | >5 | 0.215 | >5 | >5 |
| 27 | >5 | 0.022 | >5 | >5 |
| 35 | >5 | 0.054 | >5 | >5 |
| 102 | >5 | 0.340 | >5 | >5 |
| 103 | | 0.011 | | |
| 104 | | 0.125 | | |
| 105 | | 0.264 | | |
| 106 | | 11.1 | | |
| 107 | | 0.082 | | |
| 108 | | 0.277 | | |
| 109 | | 0.066 | | |
| 110 | | 0.066 | | |
| 111 | | 0.047 | | |
| 112 | | 0.068 | | |
| 113 | | 0.051 | | |
| 114 | | 1.63 | | |
| 115 | | 0.065 | | |
| 116 | | 0.943 | | |
| 117 | | 0.038 | | |
| 118 | | 0.011 | | |
| 119 | | 0.063 | | |
| 120 | | 0.018 | | |
| 121 | | 0.034 | | |
| 122 | | 0.058 | | |
| 123 | | 0.018 | | |
| 124 | | 0.032 | | |
| 125 | | 0.825 | | |
| 126 | | 0.204 | | |
| 127 | | 0.722 | | |
| 128 | | 0.055 | | |
| 129 | | 0.011 | | |
| 130 | | 0.229 | | |
| 131 | | 7.6 | | |
| 132 | | 0.176 | | |
| 133 | | 27.5 | | |
| 134 | | 10.1 | | |
| 135 | | 2.35 | | |
| 136 | | 0.077 | | |
| 137 | | 9.08 | | |
| 138 | | 0.126 | | |
| 139 | | 0.053 | | |
| 140 | | 0.364 | | |
| 141 | | 0.074 | | |
| 142 | | 0.642 | | |
| 143 | | 0.024 | | |
| 144 | | 0.011 | | |
| 145 | | 2.000 | | |
| 146 | | 0.229 | | |
| 147 | | 4.320 | | |
| 148 | | 0.074 | | |
| 149 | | 0.118 | | |
| 150 | | 0.096 | | |
| 151 | | 0.052 | | |
| 152 | | 0.409 | | |
| 153 | | 4.03 | | |
| 6 | >5 | 0.007 | 0.071 | 0.073 |
| 10 | >5 | 0.004 | NA | NA |
| 36 | >5 | 0.025 | 0.45 | 0.612 |
| 37 | >5 | 0.077 | NA | NA |
| 14 | >5 | 0.009 | 1.3 | 1.235 |
| 38 | >5 | 0.038 | 0.9 | 0.6905 |
| 39 | >5 | 0.002 | >5 | >5 |
| 22 | >5 | 0.007 | NA | NA |
| 18 | >5 | 0.012 | 0.07 | 0.102 |
| 40 | NA | 0.014 | >5 | >5 |
| 41 | NA | 0.025 | >5 | >5 |
| 42 | NA | 0.009 | >5 | >5 |
| 43 | NA | 0.605 | NA | NA |
| 44 | NA | 0.006 | >5 | >5 |
| 45 | >5 | 0.004 | >5 | >5 |
| 46 | >5 | 0.004 | >5 | >5 |
| 47 | >5 | 0.010 | >5 | >5 |
| 48 | >5 | >5 | >5 | >5 |
| 49 | >5 | >5 | >5 | >5 |
| 50 | >5 | 0.058 | >5 | >5 |
| 24 | >5 | 0.049 | >5 | >5 |
| 23 | >5 | 0.195 | >5 | >5 |
| 55 | >5 | 0.005 | >5 | 4.470 |
| 56 | 1.8 | 0.002 | >5 | >5 |
| 55 | 3.255 | 0.018 | 0.42 | 0.285 |
| 56 | NA | 0.035 | >5 | >5 |
| 57 | NA | 0.020 | 2.375 | 1.052 |
| 58 | NA | 0.005 | 1.377 | 0.871 |
| 59 | >5 | 0.036 | >5 | >5 |
| 60 | >5 | 0.017 | >5 | >5 |
| 61 | >5 | 0.012 | >5 | >5 |
| 62 | >5 | 0.024 | >5 | >5 |
| 63 | >5 | 0.360 | >5 | >5 |
| 64 | >5 | 0.035 | >5 | >5 |
| 65 | >5 | 0.042 | NA | NA |
| 66 | >5 | 0.039 | >5 | >5 |
| 67 | >5 | 0.018 | >5 | >5 |
| 68 | >5 | 0.102 | >5 | >5 |
| 69 | >5 | 0.007 | 1.540 | 2.530 |
| 70 | >5 | 0.005 | 1.520 | 1.965 |
| 71 | >5 | 0.034 | 2.039 | 3.315 |
| 72 | >5 | 0.007 | 1.510 | 4.090 |
| 73 | >5 | 0.040 | >5 | >5 |
| 74 | >5 | 0.016 | >5 | >5 |
| 75 | >5 | 0.780 | >5 | >5 |
| 76 | >5 | 0.025 | >5 | >5 |
| 77 | >5 | 0.045 | >5 | >5 |
| 78 | >5 | 0.051 | >5 | >5 |
| 79 | >5 | 0.125 | >5 | >5 |
| 80 | NA | 0.021 | >5 | >5 |
| 81 | >5 | 0.007 | >5 | >5 |
| 82 | >5 | 0.047 | >5 | >5 |
| 83 | >5 | 0.064 | >5 | >5 |
| 84 | >5 | 0.018 | >5 | >5 |
| 85 | >5 | 0.051 | >5 | >5 |
| 86 | >5 | 0.830 | >5 | >5 |
| 87 | >5 | 0.250 | >5 | >5 |
| 88 | >5 | 0.335 | >5 | >5 |
| 89 | >5 | 0.545 | >5 | >5 |
| 90 | >5 | 3.200 | >5 | >5 |
| 91 | >5 | 0.675 | >5 | >5 |
| 92 | >5 | 0.455 | >5 | >5 |
| 93 | >5 | 0.750 | >5 | >5 |
| 94 | >5 | 0.465 | >5 | >5 |
| 95 | >5 | 0.210 | >5 | >5 |
| 96 | >5 | 1.450 | >5 | >5 |
| 97 | >5 | 0.295 | >5 | >5 |
| 98 | >5 | 0.430 | >5 | >5 |
| 99 | >5 | 0.350 | >5 | >5 |
| 100 | >5 | 1.175 | >5 | >5 |

(NA = not available)

The invention and the manner and process of making and using it, are now described in such full, clear concise, and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A compound of the formula I: 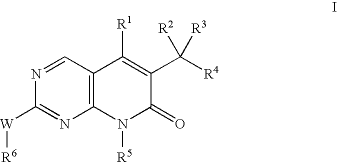
wherein:
W is selected from the group consisting of NH, N(CO)R⁷, NR⁷, S, S(O), S(O)₂, and halogen;
R¹ is hydrogen, C₁-C₆ alkyl, halogen, OR⁷, or NR⁷R⁸;
R² and R³ are independently selected from the group consisting of hydrogen, halogen, or C₁-C₆ alkyl, or R², R³ and the carbon to which they are attached may form a carbonyl group (C═O), or a C₃-C₅ carbocyclic ring;
R⁴ is a C₆-C₁₀ monocyclic or bicyclic aryl group or a 5 or 6 membered heteroaryl group wherein said aryl or heteroaryl group is optionally substituted with up to 5 substituents independently selected from halogen, C₁-C₆ alkyl, C₁-C₆ alkoxy, C₁-C₈ alkoxyalkyl, C₁-C₈ haloalkyl, C₁-C₈ hydroxyalkyl, C₂-C₈ alkenyl, C₂-C₈ alkynyl, nitrile, and nitro; wherein said heteroaryl ring is selected from pyrrolyl, thienyl, furanyl, thiazolyl, triazolyl, imidazolyl, (is)oxazolyl, oxadiazolyl, tetrazolyl, pyridyl, thiadiazolyl, oxadiazolyl, oxathiadiazolyl, thiatriazolyl, pyrimidinyl, quinolinyl, isoquinolinyl, napthyridinyl, phthalimidyl, benzimidazolyl, and benzoxazolyl;
R⁵ is hydrogen, aryl, C₁-C₈ alkyl, C₁-C₈ alkoxy, C₃-C₇ cycloalkyl, C₃-C₈ alkenyl, C₅-C₈ cycloalkenyl, C₆-C₁₀ aryl, C₅-C₁₀ heteroaryl, or C₃-C₇-heterocyclyl;
R⁶ is hydrogen, (CR⁷R⁸)ₙAr, (CR⁷R⁸)ₙheteroaryl, (CR⁷R³)ₙheterocyclyl, C₁-C₁₀ alkyl, C₃-C₁₀ cycloalkyl, C₂-C₁₀ alkenyl, or C₂-C₁₀ alkynyl, wherein each of the (CR⁷R⁸)ₙAr, (CR⁷R⁸)ₙheteroaryl, alkyl, cycloalkyl, alkenyl, and alkynyl groups are optionally substituted with up to 7 groups selected from halogen, C₁-C₈ alkyl, C₃-C₇ cycloalkyl, C₃-C₈ cycloalkyloxy, C₃-C₈ heterocyclyl, C₃-C₈ heterocycloalkyloxy, C₃-C₈ heterocyclylalkyl, C₁-C₈ alkoxy, C₁-C₈ alkoxyalkyl, C₁-C₈ haloalkyl, C₁-C₈ hydroxyalkyl, C₂-C₈ alkenyl, C₂-C₈ alkynyl, phenoxy, phenyl, (CR⁹R¹⁰)ₙAr, (CR⁹R¹⁰)ₙheteroaryl, nitrile, nitro, (CR⁷R⁸)ₘR⁹, OR⁷, SR⁷, NR⁷R⁸, N(O)R⁷R⁸, P(O)(OR⁷)(OR⁸), (CR⁷R⁸)ₘNR⁹R¹⁰, (CR⁷R⁸)ₘC(O)N⁹R¹⁰, (CR⁷R⁸)ₘOR⁹, (CR⁷R⁸)ₘC(O)R⁹, (CR⁷R⁸)ₘCO₂R⁹, CONR⁷R⁸, C(O)NR⁷SO₂R⁸, NR⁷SO₂R⁸, C(O)NR⁷OR⁸, (CR⁷R⁸)ₘS(O)ₙR⁹, SO₂NR⁷R⁸, NR⁷CO₂R⁸, NR⁷COR⁸, NR⁷CONR⁸R⁹, NR⁷SO₂R⁸, N(O)R⁷R⁸, NR⁷R⁸R⁹Y, (CR⁷R⁸)ₘP(O)(OR⁹)(OR¹⁰), -T(CH₂)ₘQR⁷, —C(O)T(CH₂)ₘQR⁷, -T(CH₂)ₘC(O)NR⁷R⁸, -T(CH₂)ₘCO₂R⁷, and NR⁷C(O)T(CH₂)ₘQR⁷; wherein R⁶ optionally may be absent when W is a halogen;
n is an integer of from 1 to 3;
m is an integer of from 0 to 5;
T is selected from the group consisting of O, S, NR⁹, N(O)R⁹, and CR⁹R¹⁰;
Q is selected from the group consisting of O, S, NR⁹, N(O)R⁹, CO₂, O(CH₂)ₘ-heteroaryl, O(CH₂)ₘS(O)ₙR⁹, (CH₂)-heteroaryl, and a saturated or unsaturated carbocyclic group containing from 3-7 ring members, up to four of which ring carbon atoms are optionally replaced by heteroatoms independently selected from oxygen, sulfur, and nitrogen, provided, however, that there is at least one carbon atom in the carbocyclic ring and that, if there are two or more ring oxygen atoms, the ring oxygen atoms are not adjacent to one another, wherein the heteroaryl or carbocyclic group is unsubstituted or substituted with one, two, or three groups independently selected from halogen, hydroxy, hydroxyalkyl, C₁-C₈ alkyl, C₁-C₈ alkoxy, alkoxycarbonyl, alkylcarbonyl, alkylcarbonylamino, aminoalkyl, trifluoromethyl, N-hydroxyacetamide, trifluoromethylalkyl, amino, and mono or dialkylamino;
R⁷, R⁸, R⁹ and R¹⁰ are, in each instance, independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl;
or R⁷ and R⁸, when attached to the same nitrogen atom, taken together with the nitrogen to which they are attached, may form a saturated or unsaturated heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁹SO₂R¹⁰, C(O)NR⁹R¹⁰, NR⁹C(O)R¹⁰, C(O)OR⁹, C(O)NR⁹SO₂R¹⁰, (CH₂)ₘS(O)ₙR⁹, (CH₂)ₘ-heteroaryl, O(CH₂)ₘ-heteroaryl, (CH₂)ₘC(O)NR⁹R¹⁰, O(CH₂)ₘC(O)OR⁹, and (CH₂)SO₂NR⁹R¹⁰;
and the pharmaceutically acceptable salts, esters, amides, and prodrugs thereof.

2. A compound of claim 1, having the following structure: 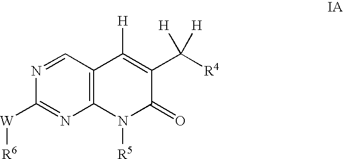
wherein:
all substituents and n are as defined as for Formula I.

3. A compound of claim 1 or claim 2 wherein W is NH and R⁴, R⁵ and R⁶ are as defined in claim 1.

4. A compound selected from:
8-Cyclopentyl-6-(5-methyl-[1,3,4]oxadiazol-2-ylmethyl)-2-(4-piperazin-1-yl-phenylamino)-8H-pyrido[2,3-d]pyrimidin-7-one;
6-Benzyl-8-cyclopentyl-2-(4-piperazin-1-yl-phenylamino)-8H-pyrido[2,3-d]pyrimidin-7-one;
6-Benzoyl-8-cyclopentyl-2-(4-piperazin-1-yl-phenylamino)-8H-pyrido[2,3-d]pyrimidin-7-one;
8-Cyclopentyl-2-(4-piperazin-1-yl-phenylamino)-6-thiazol-2-ylmethyl-8H-pyrido[2,3-d]pyrimidin-7-one;
8-Cyclopentyl-6-(5-methyl-[1,3,4]oxadiazol-2-ylmethyl)-2-methylsulfanyl-8H-pyrido[2,3-d]pyrimidin-7-one;
8-Cyclopentyl-2-(piperidin-4-ylamino)-6-pyridin-2-ylmethyl-8H-pyrido[2,3-d]pyrimidin-7-one;
6-Benzyl-8-cyclopentyl-5-methyl-2-(4-piperazin-1-yl-phenylamino)-8H-pyrido[2,3-d]pyrimidin-7-one;
8-Cyclopentyl-6-(hydroxy-phenyl-methyl)-5-methyl-2-(4-piperazin-1-yl-phenylamino)-8H-pyrido[2,3-d]pyrimidin-7-one;
6-Benzoyl-8-cyclopentyl-5-methyl-2-(4-piperazin-1 -yl-phenylamino)-8H-pyrido[2,3-d]pyrimidin-7-one;
8-Cyclopentyl-2-(4-piperazin-1-yl-phenylamino)-6-pyridin-2-ylmethyl-8H-pyrido[2,3-d]pyrimidin-7-one;
8-Cyclopentyl-2-phenylamino-6-pyridin-2-ylmethyl-8H-pyrido[2,3-d]pyrimidin-7-one;
8-Cyclopentyl-6-(3,4-dimethoxy-benzyl)-2-(4-piperazin-1-yl-phenylamino)-8H-pyrido[2,3-d]pyrimidin-7-one;
8-Cyclopentyl-2-[4-(2-oxo-oxazolidin-3-yl)-phenylamino]-6-(1-phenyl-ethyl)-8H-pyrido[2,3-d]pyrimidin-7-one;
6-Benzyl-8-cyclopentyl-2-(4-dimethylamino-cyclohexylamino)-8H-pyrido[2,3-d]pyrimidin-7-one;
6-Benzyl-2-cyclohexylamino-8-cyclopentyl-8H-pyrido[2,3-d]pyrimidin-7-one;
6-Benzyl-8-cyclopentyl-2-(2-hydroxy-ethylamino)-8H-pyrido[2,3-d]pyrimidin-7-one;
6-Benzyl-8-cyclopentyl-2-(1-propyl-piperidin-4-ylamino)-8H-pyrido[2,3-d]pyrimidin-7-one;
6-Benzyl-8-cyclopentyl-2-(piperidin-4-ylamino)-8H-pyrido[2,3-d]pyrimidin-7-one;
6-Benzyl-8-cyclopentyl-2-methylamino-8H-pyrido[2,3-d]pyrimidin-7-one;
6-Benzyl-8-cyclopentyl-2-dimethylamino-8H-pyrido[2,3-d]pyrimidin-7-one;
6-Benzyl-8-cyclopentyl-2-(5-piperazin-1-yl-pyridin-2-ylamino)-8H-pyrido[2,3-d]pyrimidin-7-one;
6-Benzyl-8-cyclopentyl-2-(1-methanesulfonyl-piperidin-4-ylamino)-8H-pyrido[2,3-d]pyrimidin-7-one;
8-Cyclopentyl-2-(4-dimethylamino-cyclohexylamino)-6-thiazol-2-ylmethyl-8H-pyrido[2,3-d]pyrimidin-7-one;
8-Cyclopentyl-2-(1-propyl-piperidin-4-ylamino)-6-pyridin-2-ylmethyl-8H-pyrido[2,3-d]pyrimidin-7-one.

5. A method of treating a disorder or condition caused by abnormal cell proliferation in a mammal comprising administering to said mammal an amount of a compound according to claim 1 that is effective in treating such condition or disorder.

6. The method of claim 5 wherein the disorder or condition being treated is selected from the group consisting of vascular smooth muscle proliferation associated with atherosclerosis, postsurgical vascular stenosis and restenosis, and endometriosis.

7. A method of treating a disorder or condition caused by infections selected from the group consisting of viral infections and fungal infections in a mammal comprising administering to a mammal in need of such treatment an amount of a compound according to claim 1 that is effective in treating such condition or disorder.

8. A method of treating disorders selected from the group consisting of autoimmune diseases selected from the group consisting of psoriasis, inflammation, lupus, type 1 diabetes, diabetic nephropathy, multiple sclerosis, glomerulonephritis, organ transplant rejection, graft versus host disease in a mammal comprising administering to a mammal in need of such treatment an amount of a compound according to claim 1 that is effective in treating such condition or disorder.

9. The method of treating neurodegenerative disorders in a mammal comprising administering to said mammal an amount of a compound according to claim 1 that is effective in treating such condition or disorder.

10. The method of claim 6 wherein the abnormal cell proliferation is a cancer selected from the group consisting of cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia.
